(19) United States
(12) Patent Application Publication
(10) Pub. No.: US 2024/0207080 A1
(43) Pub. Date: Jun. 27, 2024

(54) SYSTEMS AND MEHTODS FOR STABILIZING ANTI-MIGRATION ANCHOR SYSTEM

(71) Applicant: MetaModix, Inc., Plymouth, MN (US)

(72) Inventors: Kedar R. Belhe, Minnetoka, MN (US); Werner Schwarz, Ruhpolding (DE); Todd Stangenes, Mineapolis, MN (US); Parker Hagen, Minneapolis, MN (US); Richard Mattison, Zimmerman, MN (US)

(21) Appl. No.: 18/288,694

(22) PCT Filed: Apr. 28, 2022
(86) PCT No.: PCT/US2022/026756

§ 371 Date: Oct. 27, 2023

Related U.S. Application Data
(60) Provisional application No. 17/243,255, filed on Apr. 28, 2021.

(51) Int. Cl. A61F 5/00; A61F 2/04; A61F 2/95

(52) U.S. Cl. CPC A61F 5/0079; A61F 2/9517; A61F 5/0089; A61F 2002/045; A61F 2002/9505; A61F 2220/0008; A61F 2230/0039; A61F 2230/0091; A61F 2250/0039

(57) ABSTRACT

A system for delivering an anti-migration anchor to an implantable device includes a locator system having a head portion adapted to mate with the neck portion of the device, a locator capsule coupled to the head portion, and a plurality of arms coupled to the head portion and the locator capsule, the arms having a collapsed and an extended configuration. The system includes a catheter having a distal portion coupled to and extending from the head portion. The system includes a handle coupled to a portion of the catheter, the handle having a mechanism to effect relative motion between an inner and an outer member of the catheter to cause the arms to transition from the collapsed to the extended configuration. The system includes a plurality of elongate elements having a distal end coupled to the arm and configured to facilitate the delivery of an anti-migration anchor to the arm.

### TECHNICAL FIELD

The instant disclosure relates generally to implants placed within gastrointestinal tract, including, the stomach and the small intestine. More particularly, it relates to devices and methods for stabilizing systems having components implantable and removable using endoscopic techniques for treatment of obesity, diabetes, Non-Alcoholic Fatty Liver Disease (NAFLD), gastroparesis and other gastrointestinal conditions.

### BACKGROUND

Bariatric surgery procedures, such as sleeve gastrectomy, the Roux-en-Y gastric bypass (RYGB) and the bileo-pancreatic diversion (BPD), modify food intake and/or absorption within the gastrointestinal system to effect weight loss in obese patients. These procedures affect metabolic processes within the gastrointestinal system, by either short circuiting certain natural pathways or creating different interactions between the consumed food, the digestive tract, its secretions and the neuro-hormonal system regulating food intake and metabolism. In the last few years, there has been a growing clinical consensus that obese patients who undergo bariatric surgery see a remarkable resolution of their type-2 Diabetes Mellitus (T2DM) soon after the procedure. The remarkable resolution of diabetes after RYGB and BPD typically occurs too fast to be accounted for by weight loss alone, suggesting there may be a direct impact on glucose homeostasis. The mechanism of this resolution of T2DM is not well understood, and it is quite likely that multiple mechanisms are involved.

One of the drawbacks of bariatric surgical procedures is that they require fairly invasive surgery with potentially serious complications and long patient recovery periods. In recent years, there has been increased effort to develop minimally invasive procedures to mimic the effects of bariatric surgery. Many such procedures involve the use of gastrointestinal implants within the stomach or the small intestine that modify transport and absorption of food and organ secretions. One of the principal challenges with such procedures includes the difficulty in safely anchoring implants in the dynamic environment of the gastrointestinal tract, due to the intermittent and complex peristaltic motion within the gastrointestinal tract. Attempts have been made to secure implants within the gastrointestinal tract with means such as sutures, staples and barbs. For example, U.S. Pat. No. 7,476,256 describes an implant having a tubular sleeve with anchoring barbs, which penetrate the wall of the small intestine. However, stents with active fixation means, such as the barbs described in U.S. Pat. No. 7,476,256 that penetrate the wall of the stomach or the small intestine into surrounding tissue, may potentially cause tissue necrosis and erosion of the implants through the tissue. These systems are also associated with risks that penetrating the walls of the stomach or the small intestine establish a pathway for bacterial translocation from the non-sterile environment inside the gastro-intestinal tract into the sterile environment of the various organs in the abdominal cavity. This increases the risk of infections of the surrounding organs such as the liver and the pancreas and can pose a very serious health risk and require aggressive treatment including surgery.

### SUMMARY

According to one example (“Example 1”), a system for delivering an anti-migration anchor to a gastrointestinal device includes a proximal portion, a distal portion and a neck portion. The anti-migration anchor includes a first end for contacting the proximal portion and a tether portion for extending through a pylorus. The delivery system further includes a locator system having a head portion adapted to mate with the neck portion of the gastrointestinal device, a locator capsule coupled to the head portion, and a plurality of arms coupled to the head portion and the locator capsule, the arms having a collapsed configuration for delivery to the pylorus and an extended configuration for deployment of the anti-migration anchor. The delivery system further includes a catheter having a distal portion coupled to and extending from the head portion, the catheter including an outer member coupled to the head portion and an inner member coupled to the capsule. The delivery system further includes a handle coupled to a proximal portion of the catheter, the handle having a first knob configured to engage a guidewire and a second knob configured to effect relative motion between the inner member and the outer member of the catheter, so as to cause the arms to transition from the collapsed configuration to the extended configuration. The delivery system further includes a plurality of elongate elements having a proximal end and a distal end coupled the locator system arm, the elongate elements configured to facilitate the delivery of an anti-migration anchor to the locator system arm. Each of the locator system, the catheter, and the handle are configured to be advanced over a guidewire.

According to a second example (“Example 2”), the system of Example 1 further includes a guidewire extending through each of the locator system, the catheter, and the handle. The guidewire includes a distal coupling element for coupling to the gastrointestinal device.

According to a third example (“Example 3”), the system of Example 2 further includes wherein rotation of the first knob causes translation of the delivery system with respect to the guidewire.

According to a fourth example (“Example 4”), the system of Example 3 further includes wherein the handle includes a locking mechanism to lock the position of the handle with respect to the guidewire.

According to a fifth example (“Example 5”), the system of Example 1 further includes wherein the locator system includes three arms, each angularly spaced at an angle of about 120 degrees about a circumference of the head portion.

According to a sixth example (“Example 6”), the system of Example 1 further includes a deployment element configured to be delivered through one of the plurality of elongate elements to the locator system arm.

According to a seventh example (“Example 7”), the system of Example 6 further includes wherein the deployment element is a needle pushing mechanism adapted to advance a needle element forward through the pylorus and through the distal portion of the gastrointestinal device.

According to an eighth example (“Example 8”), the system of Example 7 further includes a suture tether coupled inside the needle element and configured to deploy after the needle element is advanced through the pylorus.

According to a ninth example (“Example 9”), the system of Example 8 further includes wherein the suture tether includes a first retaining tab for coupling with the proximal portion of the gastrointestinal device and a second retaining tab for coupling with the distal portion of the gastrointestinal device. The first and second tabs are connected by a flexible tether.

According to a tenth example (“Example 10”), the system of Example 6 further includes wherein the deployment element is a helix delivery element adapted to advance a helix anchor into the pylorus.

According to an eleventh example (“Example 11”), the system of Example 10 further includes wherein the helix anchor includes a helical portion configured to embed within the pylorus and a retaining tab for coupling with the proximal portion of the gastrointestinal device.

According a twelfth example (“Example 12”), a system for delivering an anti-migration anchor to an implantable device includes a proximal portion and a distal portion, the anti-migration anchor including a first end for contacting the proximal portion and a tether portion for extending through a tissue. The delivery system further includes a locator system having a head portion adapted to mate with the neck portion of the gastrointestinal device, a locator capsule coupled to the head portion, and a plurality of arms coupled to the head portion and the locator capsule, the arms having a collapsed configuration for delivery and an extended configuration for deployment of the anti-migration anchor. The delivery system further includes a catheter having a distal portion coupled to and extending from the head portion, the catheter including an outer member coupled to the head portion and an inner member coupled to the capsule. The delivery system further includes a handle coupled to a proximal portion of the catheter, the handle having a mechanism to effect relative motion between the inner member and the outer member of the catheter, so as to cause the arms to transition from the collapsed configuration to the extended configuration. The delivery system further includes a plurality of elongate elements having a proximal end and a distal end coupled the locator system arm, the elongate elements configured to facilitate the delivery of an anti-migration anchor to the locator system arm.

According to a thirteenth example (“Example 13”), the system of Example 12 further includes wherein the implantable device is a gastrointestinal implant and the issue is the pylorus, the implant further including a neck portion adapted to extend through the internal circumference of the pylorus.

According to a fourteenth example (“Example 14”), a system for delivering an anti-migration anchor to a gastrointestinal device includes a proximal portion, a distal portion and a neck portion, the anti-migration anchor including a first end for contacting the proximal portion and a tether portion for extending through a pylorus. The delivery system includes a locator system having a proximal linkage and a distal linkage each pivotably coupled to opposite sides of an intermediate linkage, the linkages having a first configuration for delivery to the pylorus and a second configuration for deployment of the anti-migration anchor. The delivery system further includes a catheter having a distal portion coupled to and extending from locator system. The delivery system further includes a tension element extending through the catheter element and coupled to the distal element of the locator system. The delivery system further includes a handle coupled to a proximal portion of the catheter, the handle having a mechanism to apply a tension to the tension element, so as to cause the locator system to transition from the first configuration to second configuration. The delivery system further includes an elongate element having a proximal end and a distal end coupled the proximal linkage of the locator system, the elongate element configured to facilitate the delivery of an anti-migration anchor to the locator system.

According to a fifteenth example (“Example 15”), the system of Example 14 further includes a deployment element configured to be delivered through the elongate element to the proximal linkage.

According to a sixteenth example (“Example 16”), the system of Example 15 further includes wherein the deployment element is a needle pushing mechanism adapted to advance a needle element forward through the pylorus and through the distal portion of the gastrointestinal device.

According to a seventeenth example (“Example 17”), a method for delivering an anti-migration anchor to an implantable device including a proximal portion and a distal portion, the anti-migration anchor including a first end for contacting the proximal portion and a tether portion for extending through a pylorus, includes advancing a delivery system over a guidewire to the pylorus. The delivery system includes a locator having a head portion adapted to mate with the neck portion of the gastrointestinal device, a locator capsule coupled to the head portion, and a plurality of arms coupled to the head portion and the locator capsule, the arms having a collapsed configuration for delivery and an extended configuration for deployment of the anti-migration anchor, a catheter having a distal portion coupled to and extending from the head portion, the catheter including an outer member coupled to the head portion and an inner member coupled to the capsule, a handle coupled to a proximal portion of the catheter, the handle having a mechanism to effect relative motion between the inner member and the outer member of the catheter, so as to cause the arms to transition from the collapsed configuration to the extended configuration, and a plurality of elongate elements having a proximal end and a distal end coupled the locator system arm, the elongate elements configured to facilitate the delivery of an anti-migration anchor to the locator system arm. The method further includes manipulating the mechanism on the handle to cause the arms to deploy from the collapsed configuration to the extended configuration. The method further includes advancing the anti-migration anchor through the elongate element to the locator adjacent the pylorus. The method further includes advancing the anti-migration anchor out of the delivery system and through the pylorus, such that the anti-migration system deploys. The method further includes wherein the anti-migration system includes a includes a first retaining tab for coupling with the proximal portion of the gastrointestinal device and a second retaining tab for coupling with the distal portion of the gastrointestinal device, the first and second retaining tabs connected by a flexible tether.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a cross-sectional view of a portion of the digestive tract in a human body with a gastrointestinal device positioned in the pylorus, according to some embodiments;

FIG. 2 is a schematic view of a gastrointestinal device, according to some embodiments;

FIG. 3 is a cross-sectional view of a portion of the digestive tract in a human body with a gastrointestinal device implanted in the pylorus, according to some embodiments;

FIG. 4A is a front perspective view of a suture tether, according to some embodiments;

FIG. 4B is a front perspective view of a suture tether, according to some embodiments;

FIG. 5 is a cross-sectional view of a portion of the digestive tract in a human body with a gastrointestinal device and a suture tether implanted in the pylorus, according to some embodiments;

FIG. 6 is a perspective view of a delivery system for an anti-migration device, according to some embodiments;

FIG. 7 is a perspective view of a handle of the delivery system of FIG. 6, according to some embodiments;

FIG. 8 is a cross-sectional side view of the handle of FIG. 7, according to some embodiments;

FIG. 9 is a cross-sectional side view of a locator system a delivery system, according to some embodiments;

FIG. 10 is a cross-sectional view of a portion of the digestive tract in a human body with a gastrointestinal implant delivery system in combination with a gastrointestinal device, according to some embodiments.

FIG. 11 is a cross-sectional view of a portion of the digestive tract in a human body with a gastrointestinal implant delivery system in combination with a gastrointestinal device, according to some embodiments.

FIG. 12 is a cross-section view of a portion of the delivery system, according to some embodiments.

FIG. 13 is a cross-sectional view of an external needle pushing element, according to some embodiments.

FIG. 14 is a cross-sectional view of a portion of the digestive tract in a human body with a gastrointestinal implant delivery system in combination with a gastrointestinal device, according to some embodiments.

FIG. 15 is a cross-sectional view of a portion of the digestive tract in a human body with a gastrointestinal implant delivery system in combination with a gastrointestinal device, according to some embodiments.

FIG. 16 is a cross-sectional view of a portion of the digestive tract in a human body with a gastrointestinal implant delivery system in combination with a gastrointestinal device, according to some embodiments.

FIG. 17 is a cross-sectional view of a locator system of a delivery system, according to some embodiments.

FIG. 18 is a cross-sectional view of a portion of a delivery system, according to some embodiments.

FIG. 19 is a perspective view of an anti-migration device, according to some embodiments.

FIG. 20 is a cross-sectional view of a external helix pushing element, according to some embodiments.

FIG. 21 is a cross-sectional view of a portion of the digestive tract in a human body with a gastrointestinal device in combination with an anti-migration device, according to some embodiments.

FIG. 22 is a cross-sectional view of a portion of the digestive tract in a human body with a gastrointestinal implant delivery system in combination with a gastrointestinal device, according to some embodiments.

FIG. 23 is a cross-sectional view of a portion of the digestive tract in a human body with a gastrointestinal implant delivery system in combination with a gastrointestinal device, according to some embodiments.

### DETAILED DESCRIPTION

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

The present disclosure relates to apparatuses, systems, and methods to place and remove apparatuses and systems within an anatomy of a patient. Using the apparatuses, systems, and methods disclosed herein, an implantable device may be placed (e.g., delivered and/or deployed) and/or retrieved from within the patient's anatomy. In various embodiments, such procedures are conducted endoscopically through the mouth, throat, stomach and intestine. Some examples relate to apparatuses, systems, and methods for placing and/or retrieving an implantable medical device from within the gastrointestinal tract of a patient, such as within the pyloric antrum, pylorus, duodenum and/or the jejunum of a patient. It will be appreciated that, in various examples, such medical devices may be delivered via one or more catheters.

In some instances, the apparatuses, systems, and methods disclosed herein may be used to secure a position of a medical device, such as a gastrointestinal device, within the patient's anatomy. For instance, in some examples, one or more anchoring elements may be utilized to secure a gastrointestinal device within a particular portion of the patient's stomach, and/or intestine, including the pyloric antrum, pylorus, duodenum, and/or jejunum. In various embodiments, these apparatuses and systems may be removed. For instance, the anchoring element(s) and gastrointestinal device may be removed after a designated period of time, or in response to an occurrence of one or more events.

As discussed in greater detail below, in various embodiments, an anchoring means, such as one or more anchoring elements, operates to tether a gastrointestinal implant to the pylorus at the base of the stomach. The pylorus is a muscular body that works as a sphincter by opening and closing with relaxation and contraction of circular muscles, thereby including a circular aperture at the base of the stomach, which acts as a valve. When fully open, the pylorus generally exhibits a maximum diameter of between twelve millimeters (12 mm) and thirty millimeters (30 mm).

Thus, the disclosed systems, devices, and methods do not penetrate from within the digestive tract into the abdominal cavity, thereby minimizing risks of bacterial translocation and subsequent infection. In various examples, the delivery system is operable to deliver a suture tether through the muscular portion of the pylorus which is contained within the non-sterile environment of the gastrointestinal tract. In some examples, the delivery system additionally includes one or more features and/or attributes that operate to minimize a risk of penetrating the sterile environment of the surrounding abdominal cavity. In some examples, the delivery system additionally includes one or more features that operate to minimize or otherwise protect the pylorus from excessive forces that could cause tears, pressure necrosis or ulceration.

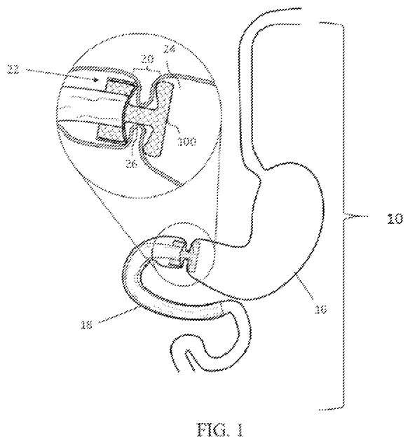

FIG. 1 shows a cross-sectional view of a portion of a human digestive tract 10, showing a stomach 16, intestine 18, the pylorus 20, and the duodenum 22. The pylorus generally includes the pyloric antrum 24 and the pyloric sphincter 26. As shown in FIG. 1, a gastrointestinal device 100 may be positioned between the stomach 16 and the intestine 18. In some examples, the gastrointestinal device 100 is positioned within the pylorus 20 such that one or more portions of the gastrointestinal device 100 are positioned within or adjacent to the pyloric antrum 24. In some examples, the gastrointestinal device 100 is additionally or alternatively positioned with one or more portions of the gastrointestinal device 100 positioned within the duodenum 22.

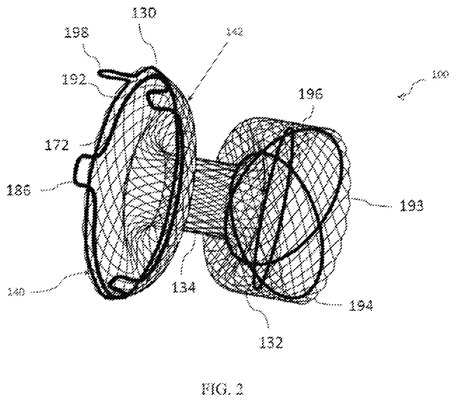

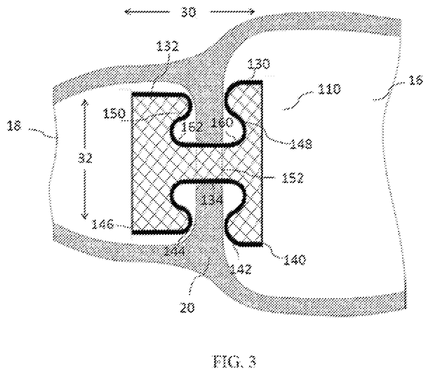

FIGS. 2 and 3 show a gastrointestinal device 100 according to various embodiments. FIG. 2 is a perspective view of the gastrointestinal device 100. FIG. 3 is a cross section view of a simplified form of the gastrointestinal device 100 in a patient's anatomy, illustrated with the proximal and distal structural elements 172 and 196 removed for clarity.

In various embodiments, the gastrointestinal device 100 is an expandable, endoscopically deliverable component that interfaces with native anatomy within the gastrointestinal tract to help effectuate weight loss. In some examples, the gastrointestinal device 100 is expandable, as those of skill will appreciate. That is, in various embodiments, upon deployment, the gastrointestinal device 100 can transition from a compressed or collapsed delivery configuration to an expanded deployed configuration. Although not shown in FIG. 2 or 3, it will be appreciated that, in various examples, a sleeve may be attached or may be attachable to the gastrointestinal device 100, upon deployment, the sleeve 120 may be positioned or positionable within the intestine 18 of the patient, as those of skill will appreciate. Thus, in various examples, the bypass sleeve may be an intestinal bypass sleeve, an intestinal liner, or a bypass liner.

With continued reference to FIGS. 2 and 3, the gastrointestinal device 100 has a generally cylindrical shape. In some embodiments, the gastrointestinal device 100 defines a central longitudinal axis along a length of the gastrointestinal device 100. The gastrointestinal device 100 also generally includes a proximal portion 130, a distal portion 132, and a neck portion 134. In various examples, the neck portion 134 is situated between the proximal portion 130 and the distal portion 132. The neck portion 134 may be integral with the proximal and distal portions 130 and 132 or may alternatively be coupled to the proximal and distal portions 130 and 132. In various examples, the neck portion 134 fluidly couples the proximal and distal portions 130 and 132. In some examples, the neck portion 134 is tubular and includes a lumen therethrough. In some such examples, the lumen extends along the longitudinal axis of the gastrointestinal device 100.

In some examples, the proximal portion 130 includes a proximal end 140, and a distal end 142. In some examples, the proximal portion 130 is cylindrical or tubular shaped. In some embodiments, a proximal wall flange 148 is situated between the proximal portion 130 and the neck portion 134. In some examples, a diameter of an outer surface of the proximal portion 130 is larger than a diameter of an outer surface of the neck portion 134. Thus, in various examples, the proximal wall flange 148 is generally disk-shaped and extends between the neck portion 134 and the proximal portion 130, as shown. In some examples, the proximal wall flange 148 is oriented transverse to the central longitudinal axis of the gastrointestinal device 100.

In various embodiments, one or more of the proximal portion 130 and the proximal wall flange 148 adopt a curved profile or are otherwise predisposed to have a curved profile when deployed (e.g., when the gastrointestinal device 100 is expanded). For instance, in some examples, one or more of the proximal portion 130 and the proximal wall flange 148 include a concavity. For example, the proximal wall flange 148 may resemble a bowl. In some other examples, one or more of the proximal portion 130 and the proximal wall flange 148 additionally or alternatively include a convexity.

In some embodiments, the distal portion 132 includes a proximal end 144, a distal end 146 and an outer wall extending in between proximal and distal ends 144 and 146. In some examples, the distal portion 132 is shaped as a flange. In some examples, the distal portion 132 is cylindrical. In some examples, a distal wall flange 150 is situated between the distal portion 132 and the neck portion 134. In some examples, a diameter of an outer surface of the distal portion 132 is larger than the diameter of the outer surface of the neck portion 134. Thus, in various examples, the distal wall flange 150 is generally disk-shaped and extends between the neck portion 134 and the distal portion 132, as shown. The distal wall flange 150 generally extends from the proximal end 144 of the distal portion 132. In some examples, the distal wall flange 150 extends transverse to the central longitudinal axis of the gastrointestinal device 100. As discussed in greater detail below, when positioned within a patient, in an expanded configuration, the distal portion 132 may be located in the duodenum, and/or may define an opening at the distal end 146 that faces the intestine 18.

The neck portion 134 includes a first end 160, a second end 162 and a wall extending between the first and second ends 160, 162. The neck portion 134 may be shaped as a cylinder that extends between the proximal portion 130 and the distal portion 132, as mentioned above. In some examples, the neck portion 134 defines a through-lumen 152 that allows contents of the stomach 16 (e.g., chime) to pass into the intestine 18. The neck portion 134 may be rigid to hold the pylorus 20 open or it may be compliant to allow the opening and closure of the through-lumen 152 with the pylorus 20.

In some embodiments, the length of the neck portion 134 may be approximately the width of a patient's pylorus. In some embodiments, the length of the neck portion 134 may be longer than the width of a patient's pylorus to provide a gap between the proximal wall flange 148, the distal wall flange 150 and the pylorus 20. In some embodiments, the neck portion 134 may be sized to allow the proximal wall flange 148 and the distal wall flange 150 to contact the pylorus 20.

In various embodiments, the gastrointestinal device 100 may be formed from a braided wire structure, as those of skill will appreciate. Such braided wire structure may help position the gastrointestinal device 100 within a patient. For example, the braided wire structure may provide structural support to the gastrointestinal device 100 and help maintain the shape of the gastrointestinal device 100.

In some embodiments, the gastrointestinal device 100 includes a structural element contained within the braided wire structure. As shown in FIG. 2, in some embodiments, the gastrointestinal device 100 has a distal structural element 196. In some examples, the distal structural element 196 is comprised of rings 193, 194 attached to the distal portion 132 and/or the neck portion 134. In some examples, the distal structural element 196 includes a metal such as Nitinol (nickel-titanium alloy), a nickel-cobalt base alloy such as that sold under the tradename MP35N®, a cobalt alloy such as Alloy L605, a cobalt-chromium-nickel-molybdenum alloy such as that sold under the tradename Elgiloy®, stainless steel, or from a plastic such as PET, PEEK, a polyoxymethylene such as that sold under the tradename Delrin® or any other suitable material. In some examples, the distal structural element 196 includes a superelastic Nitinol wire formed into a suitable shape. In an exemplary embodiment, a distal structural element 196 is formed from three rings of Nitinol wire. If the distal structural element 196 is desired with a certain rigidity or stiffness, the size and material that the distal structural element 196 is made from can be used to control these properties. For example, Nitinol wire can be used to form stiffening elements with a suitable compressive and expansive strength as a function of the diameter of the wire used to make the distal structural element 196.

As shown in FIG. 2, the rings 193, 194 of the distal structural element 196 are arranged around the distal portion 132 and are attached to the distal portion 132, such as by being integrally woven into the flange material. The rings 193, 194 of the distal structural element 196 are attached by weaving the rings 193, 194 though the braided structure of the distal portion 132. In some examples the wire ends can be inserted into a connection sleeve and crimped, welded, and/or fastened by any other suitable known means.

As shown in FIG. 2, in some embodiments, the gastrointestinal device 100 additionally or alternatively includes a proximal structural element 172 attached to the proximal portion 130. In some examples, the proximal structural element 172 is a compression biasing element, such as a spring. The proximal structural element 172 may be constructed as a substantially circular frame having nodes 186. The proximal structural element 172 may be constructed from the same material that forms the distal structural element 196. The proximal structural element 172 may also provide structural support to the proximal portion 130. For example, the proximal structural element 172 generally has an overall frame that is compressible, yet also is rigid. The proximal structural element 172 may impart additional radial strength to the proximal portion 130 and help keep the proximal end 140 of the proximal portion 130 open. The proximal structural element 172 can be shaped to bias the direction of collapse of the gastrointestinal device 100 for removal from a patient and for loading the device onto a delivery catheter for delivery within a patient.

As shown in FIG. 2, in some embodiments, the gastrointestinal device 100 may include a drawstring 192. In some examples, the drawstring 192 is attached to the proximal portion 130. The drawstring 192 can be attached to the proximal portion 130 by weaving the drawstring 192 through the material of the proximal portion 130. In various examples, the drawstring may be weaved through the material of the proximal portion and have a portion of the drawstring forming a loop 198. For example, the drawstring 192 may be constructed from a string or suture that is weaved through alternating cells in the braided wire structure of the gastrointestinal device 100. The loop 198 allows the drawstring 192 to be attached to a retraction tool, for example, to a hook or a clamp. In some embodiments, the drawstring 192 is a suture that is weaved through the proximal portion 130. The drawstring 192 may be a separate structure from the proximal structural element 172. The drawstring 192 may be constructed from a suture material and may comprise a thin wire or cable.

Turning back now to FIG. 3, when deployed within the patient's anatomy, the proximal portion 130 is generally located on the side of the pylorus 20 that is adjacent the stomach 16, with the distal portion 132 generally located on the side of the pylorus 20 that is adjacent the duodenum 22, and with the neck portion 134 spanning the pyloric sphincter 26.

As shown the gastrointestinal device 100 is deployed such that the pyloric sphincter 26 and associated tissue is sandwiched between or otherwise situated between the proximal and distal portions 130 and 132 of the gastrointestinal device 100. Conventional designs have traditionally relied on the integrity and geometry of the implanted device to resist migration and or rotation of the implanted device relative to the pylorus 20 and surrounding tissue.

For instance, some conventional devices have sought to resist or minimize rotation and migration after implantation by increasing a length and/or diameter of the portion of the device projecting into the duodenum. Such configurations provide that the device may contact the duodenum and prevent further rotation before becoming deflected or dislodged. For instance, the length and diameter of the portion of the device extending into the duodenum can be sized to prevent canting or tilting within the duodenum. In some embodiments, such configurations provide that upon rotation or canting of the device away relative to the surrounding anatomy, the device will make contact with the intestinal wall and therefore will resist migration further rotation or canting. Some other conventional designs have included active fixation means, such as barbs that deeply penetrate into surrounding tissue. However, as mentioned above, such configurations bear a risk for tissue necrosis and erosion, which can lead to complications, such as bacterial infection of the mucosal tissue or systemic infection.

In some cases, devices have included additional structural components to assist in anchoring the device to the surrounding anatomy, like those structural elements discussed above (e.g., proximal and distal structural elements 172 and 196). These structural elements, however, do not penetrate the surrounding tissue, and thus rely on the geometry of the device and its interference with the surrounding tissue to maintain alignment of the device within the anatomy.

In various embodiments, one or more suture tethers can be utilized in combination with the gastrointestinal device 100 to secure the gastrointestinal device 100 to the surrounding tissue. As explained in greater detail below, the one or more suture tethers operate to secure the gastrointestinal device to the surrounding anatomy, and, in some instances, operate to help maintain a geometry of the gastrointestinal device 100. In various examples, one or more of the suture tethers extend through one or more portions of the gastrointestinal device 100 and through one or more portions of the surrounding anatomy. Generally, the suture tethers thus operate as secondary anchoring mechanisms that help maintain a position of the gastrointestinal device 100 relative to the surrounding anatomy.

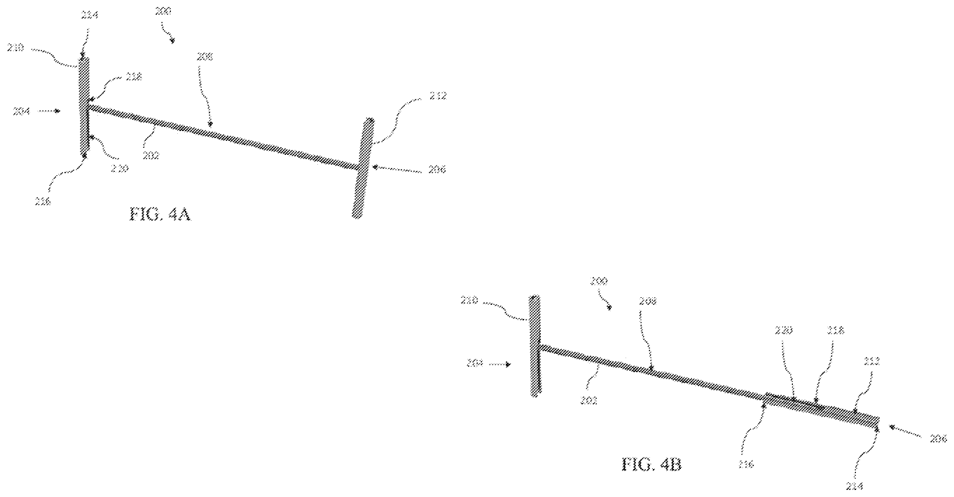

Turning now to FIGS. 4A and 4B, in some embodiments, a suture tether 200 is an anti-migration device that includes a body 202 having a first end 204, a second end 206 opposite the first end 204, and an elongate middle portion 208 extending between the first end 204 and the second end 206. The body 202 may be comprised of one or more filamentary members, a braided fiber, or may be a wire or a braided wire. That is, in some examples, the body 202 may be structurally compressible, while in other examples, the body 202 is unable to independently support a compressive load without significant deformation (e.g., folding or wrinkling). In various embodiments, the body 202 is flexible. In various examples, the body 202 is resilient to tensile loads. In some examples, the body 202 is stretch resistant. It is to be appreciated that the body could be composed of bio-compatible non-absorbable suture materials as polypropylene, PTFE, ePTFE or dPTFE, polyester, nylon, UHMWPE or stainless steel. In some examples, the body 202 is formed of a material configured to resist tissue ingrowth such as polypropylene or nylon, dPTFE or stainless steel.

In various embodiments, the suture tether 200 includes one or more retaining tabs. For example, as shown in FIG. 4A, the suture tether 200 includes a first retaining tab 210 and a second retaining tab 212. The retaining tabs 210 and 212 operate to maintain a position of the suture tether 200 relative to the gastrointestinal device 100. For example, in some instances, the retaining tabs 210 and 212 operate to minimize the risk of the suture tether 200 decoupling from the gastrointestinal device 100, as discussed further below. Though a variety of retaining tabs are contemplated and may be utilized without departing from the spirit or scope of the disclosure, in some examples, the retaining tabs are formed from one or more tubes. In some examples, as discussed in greater detail below, the tubes are configured such that the body 202 can be received within and coupled with the tube. In some examples, the tube can be crimped to facilitate a couple between the retaining tab and the body 202. The retaining tabs may be formed from variety of biocompatible materials including, but not limited to, metallics such as stainless steel and Nitinol, and polymers such as polytetrafluoroethylene (PTFE), expanded polytetrafluoroethylene (PTFE), polyether ether ketone (PEEK), ultra-high-molecular-weight polyethylene (UHMWPE), polyurethanes, polyesters, polyimide, nylon and polypropylene.

In some examples, the retaining tabs are integral with the body 202. In some such examples, the retaining tabs and the body 202 for a monolithic unit. In some examples, one or more of the retaining tabs are coupled to the body 202. In some examples, the body 202 terminates at or within a retaining tab at each of its respective ends. It will be appreciated that any suitable method may be employed to couple the retaining tabs to the body 202, including, but not limited to, clamping, gluing, pinning, tying, or utilizing one or more fastening means, as those of skill will appreciate. As shown, the retaining tabs 210 and 212 are crimped onto the body 202.

In various embodiments, the suture tether 200 is configured to transition from a delivery configuration to a deployed configuration such that the suture tether 200 can be delivered to a target region in a minimal profile and subsequently deployed (e.g., coupled with the gastrointestinal device 100) in a manner that minimizes a potential for the retaining tab to decouple from the gastrointestinal device 100. Generally, when transitioned to the deployed configuration, one or more of the retaining tabs of the suture tether 200 change shape and/or orientation relative to the body 202. In various examples, one or more of the retaining tabs 210 and 212 are coupled to the suture tether 200 such that the retaining tabs are biased to adopt the deployed configuration when unconstrained. Such a configuration provides that the one or more of the first and second retaining tabs 210 and 212 will adopt or otherwise naturally transition to the deployed configuration upon being deployed within the anatomy. In some examples, naturally transitioning to the deployed configuration upon deployment can be accomplished by creating a pre-formed bend in the body 202.

FIG. 4B shows the suture tether 200 in a partially deployed configuration to illustrate one non-limiting example of the first and second retaining tabs 210 and 212 in delivery and deployed configurations. Specifically, in FIG. 4B, the first retaining tab 210 is oriented in a deployed configuration, while the second retaining tab 212 is oriented in a delivery configuration. As shown, the orientation of the first retaining tab 210 is different than the orientation of the second retaining tab 212. In some examples, in the deployed configuration, the retaining tabs are oriented transverse to (or otherwise extends transverse to) the body 202. For example, as shown in FIG. 4B, the first retaining tab 210 (which is illustrated in the deployed configuration), extends transverse to the body 202. In some examples, in the delivery configuration, the retaining tabs extend generally along or in line with the body 202. For example, as show in FIG. 4B, the second retaining tab 212 (which is illustrated in the delivery configuration), extends generally along or in line with the body 202.

Additionally, as shown in FIG. 4A, the body 202 terminates into or is otherwise coupled to the retaining tabs 210 and 212 at a midsection thereof. For example, as shown in FIG. 4A, the body 202 terminates into the first retaining tab 210 at a midsection 218 between a first end 214 and a second end 216 of the first retaining tab 210. Accordingly, in various examples, the retaining tabs are coupled to the body 202 such that two or more portions of the retaining tabs project away from the body 202. As shown in FIG. 4A, first end 214 and second end 216 extend or project away from the body 202 in the deployed configuration.

As mentioned above, the suture tether 200 maintains a minimal profile in the delivery configuration. In some examples, the retaining tabs include one or more features to help facilitate a minimal delivery profile. For example, as shown in FIG. 4A, the first retaining tab 210 includes a relief 220 formed in the first retaining tab 210 that is configured to accommodate the body of suture tether 200 in the delivery configuration. For instance, as shown in FIG. 4B, the body 202 is accommodated by the relief 220 in the second retaining tab 212 in the delivery configuration.

It will be appreciated that while the retaining tabs 210 and 212 are illustrated in the above-discussed embodiments and examples as changing orientation relative to the body 202, in various embodiments, the retaining tabs of the suture tether 200 may additionally or alternatively change size and/or shape when the suture tether 200 is transitioned from the delivery configuration to the deployed configuration. For example, in some instances, the retaining tabs are inflatable members. In some other examples, the retaining tabs are expandable members that expand from a delivery profile to a deployed profile. In some such examples, the retaining members are self-expanding. In some examples, the retaining tabs are disc-shaped. In some examples, the retaining tabs include one or more petals that are configured to project away from the body 202 in the deployed configuration. It will be appreciated that any suitable configuration for the retaining members may be utilized provided that the retaining members transition to a deployed configuration that minimizes a potential for the retaining tabs to decouple from the gastrointestinal device 100.

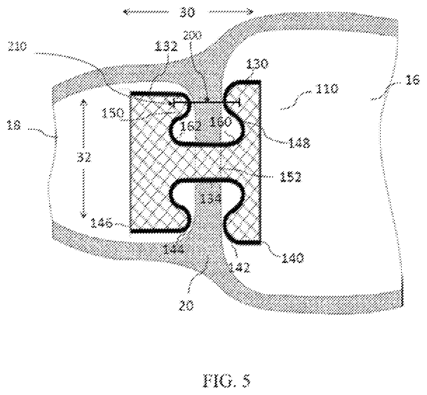

FIG. 5 is the cross section view of FIG. 3, illustrated with a deployed suture tether 200. As shown, in various examples, the suture tether 200 is configured to extend from the proximal portion 130 of the gastrointestinal device 100 to the distal portion 132 of the gastrointestinal device 100. In some examples, the suture tether 200 is configured to penetrate one or more of the proximal and distal portions 130 and 132 of the gastrointestinal device 100, as well as one or more portions of the surrounding anatomy. As discussed in greater detail below, the suture tether 200 is deployed within the anatomy such that it penetrates the pyloric sphincter 26 (e.g., the muscle associate with the pylorus 20).

As shown in FIG. 5, the suture tether 200 penetrates the proximal portion 130 of the gastrointestinal device 100. In some examples, the suture tether 200 penetrates the proximal wall flange 148 of the proximal portion 130. In some examples, the second retaining tab 212 is positioned adjacent the proximal wall flange 148 on the stomach-side of the gastrointestinal device 100. That is, in various examples, the suture tether 200 is deployed such that the proximal wall flange 148 of the proximal portion 130 (or the proximal portion 130, generally) is situated between the second retaining tab 212 and the tissue of the pylorus 20 (e.g., the pyloric sphincter 26) sandwiched between the proximal and distal portions 130 and 132 of the gastrointestinal device 100.

Similarly, as shown in FIG. 5, the suture tether 200 penetrates the distal portion 132 of the gastrointestinal device 100. In some examples, the suture tether 200 penetrates the distal wall flange 150 of the distal portion 132. In some examples, the first retaining tab 210 is positioned adjacent the distal wall flange 150 on the duodenum-side of the gastrointestinal device 100. That is, in various examples, the suture tether 200 is deployed such that the distal wall flange 150 of the distal portion 132 (or the distal portion 132, generally) is situated between the first retaining tab 210 and the tissue of the pylorus 20 (e.g., the pyloric sphincter 26) sandwiched between the proximal and distal portions 130 and 132 of the gastrointestinal device 100.

In various examples, the suture tether 200 is deployed such that suture tether 200 spans between the proximal and distal portions 130 and 132 without penetrating the neck portion 134 of the gastrointestinal device 100. For example, as shown in FIG. 5, the suture tether 200 is deployed such that it penetrates each of the proximal and distal portions 130 and 132 to the gastrointestinal device 100 and spans therebetween without penetrating the neck portion 134 of the gastrointestinal device 100. Put differently, in various examples, the suture tether 200 is deployed and penetrates the gastrointestinal device 100 at one or more positions radially outward from the neck portion 134.

It will be appreciated that one or more suture tethers 200 may be utilized to secure the gastrointestinal device 100 to the surrounding anatomy. For instance, in some examples, three suture tethers 200 may be deployed to secure the gastrointestinal device 100 to the surrounding anatomy. In some such examples, the suture tethers 200 are generally evenly distributed about the gastrointestinal device 100. For example, where three suture tethers 200 are employed to secure the gastrointestinal device 100 to the surrounding anatomy, the suture tethers 200 each may be situated 120 degrees apart.

It will also be appreciated that such a configuration provides that the one or more suture tethers 200 will operate to minimize rotation of the gastrointestinal device 100 about a longitudinal axis of the gastrointestinal device 100 in-situ, as well as migration of the gastrointestinal device 100 relative to the pylorus 20.

It has been discovered that one of the factors contributing to dislodgment and migration of gastrointestinal implants (and those situated in the pylorus in particular) involves relative angulation of the portions of the gastrointestinal implants on either side of the pyloric sphincter 26 as a result of natural contractions and movements of the surrounding tissue. For instance, as an angulation of the distal portion 132 and/or the neck portion 134 increases relative to the proximal portion 130, the gastrointestinal device 100 deforms and loses its ability to adequately conform to the anatomy of the pylorus 20. This conformability issue results in a decrease in the surface area of the proximal portion 130 reacting against or otherwise engaging the anatomy of the pylorus 20 adjacent the proximal portion 130, thereby reducing the ability of the gastrointestinal device 100 to resist dislodgment and migration. Given a sufficient amount of angulation in combination with the natural contractions and movements of the surrounding anatomy, the effective surface area of the gastrointestinal device 100 will be insufficient to sustain retention of the gastrointestinal device 100 within the pylorus 20, and the gastrointestinal device 100 will become dislodged.

The suture tether 200 thus operates as a secondary anchoring mechanism that functions to minimize a relative angulation of the proximal and distal portions 130 and 132 (and/or the neck portion 134) relative to one another, and/or relative to the surrounding anatomy. The suture tether 200 physically secures the gastrointestinal device 100 to the surrounding anatomy by penetrating the surrounding anatomy and one or more of the proximal and distal portions 130 and 132 of the gastrointestinal device 100. In some such examples, the suture tether 200 operates to maintain a relative alignment of the anatomy and the portion of the gastrointestinal device 100 to which the suture tether 200 is coupled. In some examples, such a configuration operates to maximize and maintain the effective surface area of the gastrointestinal device 100 available for reacting against or otherwise engaging the surrounding anatomy to prevent dislodgment and/or migration.

In those configurations where the suture tether 200 extends through the surrounding anatomy and each of the proximal and distal portions 130 and 132 of the gastrointestinal device 100, the suture tether 200 additionally operates to minimize the amount of relative angulation between the proximal and distal portions 130 and 132 of the gastrointestinal device 100, thereby minimizing the amount of deformation of the gastrointestinal device 100. By further minimizing the amount of deformation of the gastrointestinal device 100 the suture tethers 200 operate to maximize and maintain the effective surface area of the gastrointestinal device 100 available for reacting against or otherwise engaging the surrounding anatomy to prevent dislodgment and/or migration.

In various examples, the amount to which the proximal and distal portions 130 and 132 are free to angulate relative to one another is based, at least in part, on a length of the suture tethers 200 relative to a distance between the proximal and distal portions 130 and 132, as those of skill will appreciate. In some examples, a length of the suture tether 200 (e.g., a distance between the first and second retaining tabs 210 and 212) exceeds a distance between proximal and distal wall flanges of a given gastrointestinal device such that the first and second retaining tabs 210 and 212 do not contact tissue when implanted or pinch the proximal and distal wall flanges of a given gastrointestinal device together. For instance, in some nonlimiting examples, a distance between proximal and distal wall flanges of a gastrointestinal device may be eleven millimeters, while a distance between the first and second retaining tabs 210 and 212 of the suture tether 200 a may be between fifteen and thirty millimeters. In some examples, selecting or configuring the suture tethers in such a manner helps avoid pressure necrosis, ulceration and other damage to the anatomy. Moreover, selecting or configuring the suture tethers to have a length that exceeds a distance between proximal and distal wall flanges of a given gastrointestinal device allows for the gastrointestinal device to dynamically adjust to the anatomy as the surrounding anatomy moves in association with digestive behavior. The longer a given suture tether 200 is in relation to the distance between the proximal and distal portions 130 and 132, the greater the amount of potential angulation between the proximal and distal portions 130 and 132. Some degree of angulation between the proximal and distal portions 130 and 132 may be desired. For instance, some degree of angulation between the proximal and distal portions 130 and 132 may provide for a gastrointestinal device 100 that more appropriately conforms to the surrounding anatomy.

Examples of suitable constructions of the gastrointestinal device 100 are illustrated and described in U.S. patent application Ser. Nos. 15/060,418, 14/872,990, and 15/600,214, the contents of each of which are incorporated herein by reference. It will be appreciated that the gastrointestinal device 100 may be delivered according to methods known to those of skill in the art. Examples of suitable methods for delivering the gastrointestinal device 100 are illustrated and described in U.S. patent application Ser. Nos. 15/060,418, 14/872,990, and 15/600,214, mentioned above.

In various examples, the gastrointestinal device 100 may include one or more anchoring components that individually or collectively operate to maintain a position of the gastrointestinal device 100 within the patient's anatomy. In some examples, a sleeve 120 may be attached to the anchor 110, as those of skill will appreciate. It will also be appreciated that the gastrointestinal device 100 may be an implant, a gastrointestinal implant, or a pyloric implant.

In various embodiments, the gastrointestinal device 100 and the suture tethers 200 may be endoscopically implanted within and/or retrieved from the patient's anatomy while in a delivery configuration as discussed above. Generally, in the delivery configuration, the gastrointestinal device 100 and/or the suture tethers 200 are in a closed, compressed, or collapsed configuration in that they possess a smaller profile than a deployed profile, as will be appreciated.

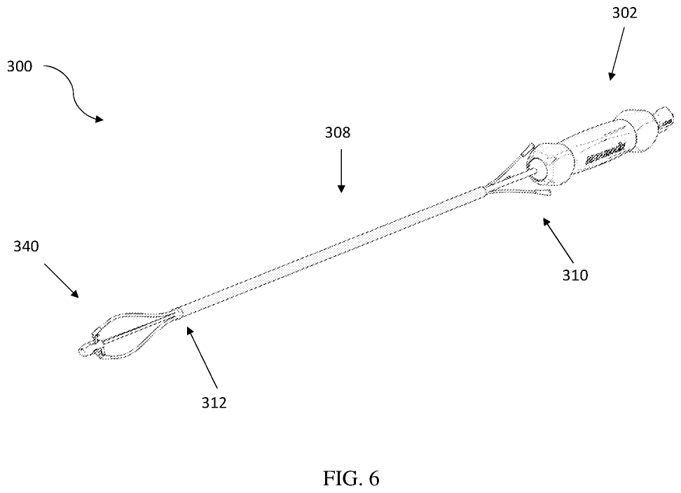

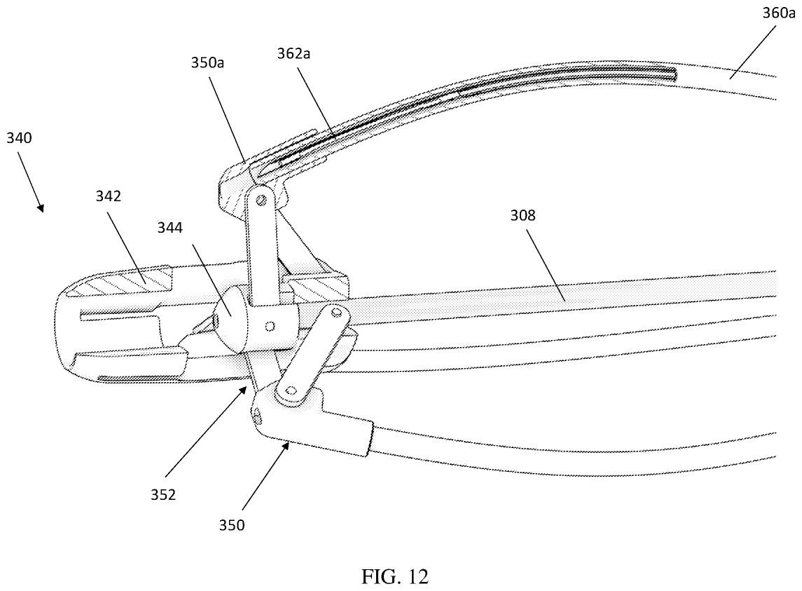

FIG. 6 is a perspective view of a delivery system 300 for delivering an anti-migration device into a patient's anatomy, such as the delivery of the suture tether 200 (FIG. 4A). The delivery system 300 may comprise a handle 302, a catheter 308 and a locator system 340. In some examples, the locator system 340 is situated at or proximate a distal end 312 of the catheter 308. In some examples, the catheter 308 includes a through lumen for accommodating a guidewire (GW), (not shown). In various embodiments, the locator system 340, the catheter 308, and the handle 302 are configured to be advanced over the guidewire (not shown). The delivery system 300 is illustrated in FIGS. 6-9 without a corresponding gastrointestinal device or a surrounding anatomy to more clearly depict the elements and features of the delivery system 300. Operation of the delivery system 300 with a gastrointestinal device and within a portion of the digestive tract of a patient is shown and described below in relation to at least FIGS. 12-16.

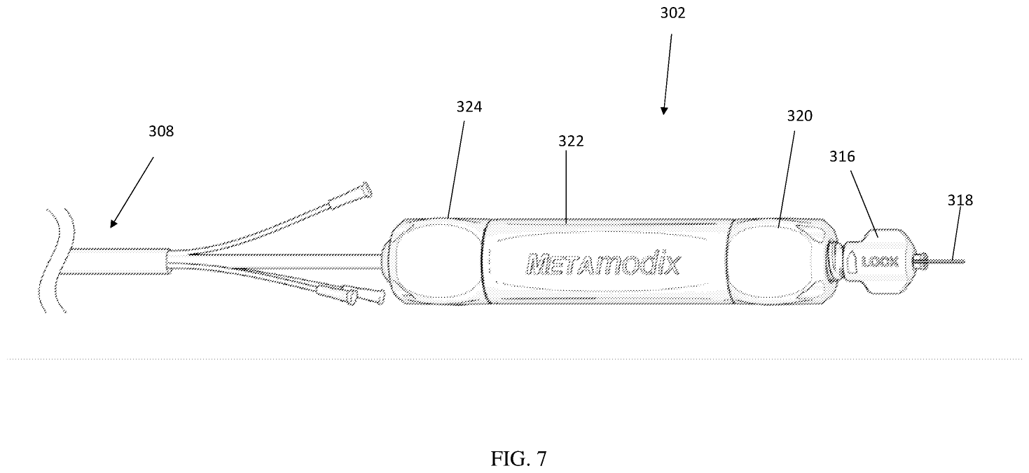

FIG. 7 is a perspective view of the handle 302 of the delivery system 300. As shown in FIG. 7, the handle 302 includes a locking element 316 configured for locking the guidewire 318 in place, a first knob 320 configured for tensioning or translating the guidewire 318 and a second knob 324 configured changing a locator system (not shown) to a deployed system through a translation of a carriage 332. In various embodiments, the handle 302 is operably coupled with the catheter 308. The handle 302 may be coupled to the proximal end 310 of the catheter 308. As previously described, the catheter 308 may accommodate the guidewire 318. In various examples, the catheter 308 includes a lumen extending therethrough. In various examples, the guidewire 318 is coaxially received within the lumen of the catheter 308. In various examples, the locator system 340 is configured to engage or otherwise interface with a gastrointestinal device that has been deployed within the anatomy. The engagement between the locator system 340 and the deployed gastrointestinal device helps maintain or constrain an orientation of the delivery system 300 while the suture tethers 200 are delivered and deployed.

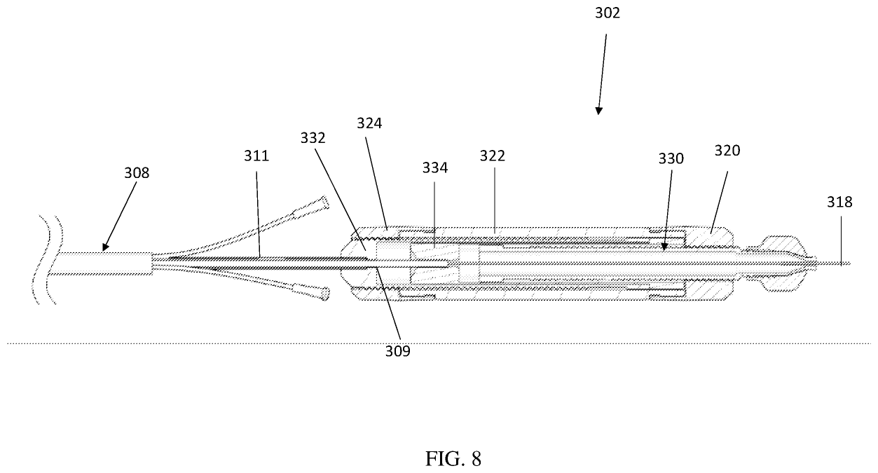

FIG. 8 is a cross-sectional view of the handle 302 illustrated in FIG. 7. The catheter 308 is shown with the handle 302 extending from the proximal end 310 of the catheter 308. The catheter 308 includes an inner member 309 and an outer member 311. The inner member 309 and outer member 311 may be referred to as an inner catheter 309 and an outer catheter 311 herein. The outer member 311 is coupled to the carriage 332. The inner member 209 is coupled to a translation portion 334. The coupling of the inner member 309 to translation portion 334 and the outer member 311 to carriage 332 allows for the operator to actuate the locator system (not shown) to the deployed configuration through the actuation of the second knob 324. Within the handle 302, the guidewire 318 extends through the inner member 309, through the handle 302, and outward from the proximal end of the handle 302. As shown and previously mentioned with respect to FIG. 7, the handle 302 includes the first knob 320 positioned at a proximal end of the handle 302, and the second knob 324 positioned at a distal end of the handle 302. The handle 302 further includes a body portion 322 positioned between the first knob 320 and the second knob 324 with the body portion 322 operatively coupled to each the first knob 320 and the second knob 324, and further includes a carriage 332 operatively coupled to the handle 302 and configured to translate within the handle 302. Further, the first knob 320 has a grooved surface and engages with a guidewire housing 330 and the second knob 324 has a grooved surface and engages with the carriage 332, such that rotation of the first knob 320 causes translation of the guidewire 318 and rotation of the second knob 324 causes translation of the carriage 332.

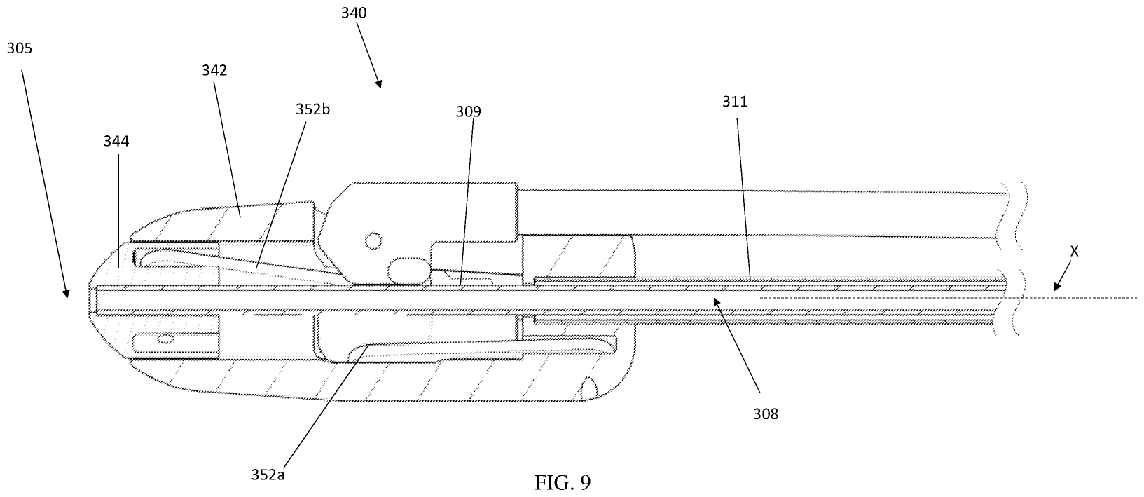

FIG. 9 is a cross sectional view of the locator system 340 the delivery system 300. As illustrated in FIG. 9, the locator system 340 includes a locator capsule 342, a head portion 344, and a plurality of linking arms 352 coupled to the head portion 344. The locator capsule 342 has a body that is shaped and sized in a manner that is complimentary to a shape and size of the deployed gastrointestinal device. Thus, in some examples, the locator capsule 342 may be cylindrically shaped and configured to be received within a through-lumen of the deployed gastrointestinal device to cause engagement between the locator capsule 342 and the gastrointestinal device. In various embodiments, the locator capsule 342 is configured to interface with or engage the deployed gastrointestinal device in a number of other suitable manners, provided that the engagement between the locator capsule 342 and the gastrointestinal device operates to maintain or constrain an orientation of the delivery system 300 while the suture tethers 200 are delivered and deployed. As illustrated in FIG. 9, the outer member 311 of the catheter 308 is coupled to the proximal end of the locator capsule 342. The inner member 309 of the catheter 308 is coupled to the head portion 344 of the locator capsule 342. As a result of this configuration, the head portion 344 includes the ability to translate along a longitudinal axis X of the locator system 340 when actuated through the rotation of the second knob 324 of previously described handle 302, and operation of the plurality of linking arms 352, as will be described further herein. The head portion 344 additionally includes an opening for receiving the catheter 308 of the delivery system 300 (FIG. 6). Thus, the guidewire 318 is housed within the catheter 308 and capable of extending outward from the head portion 344 of the locator system 300.

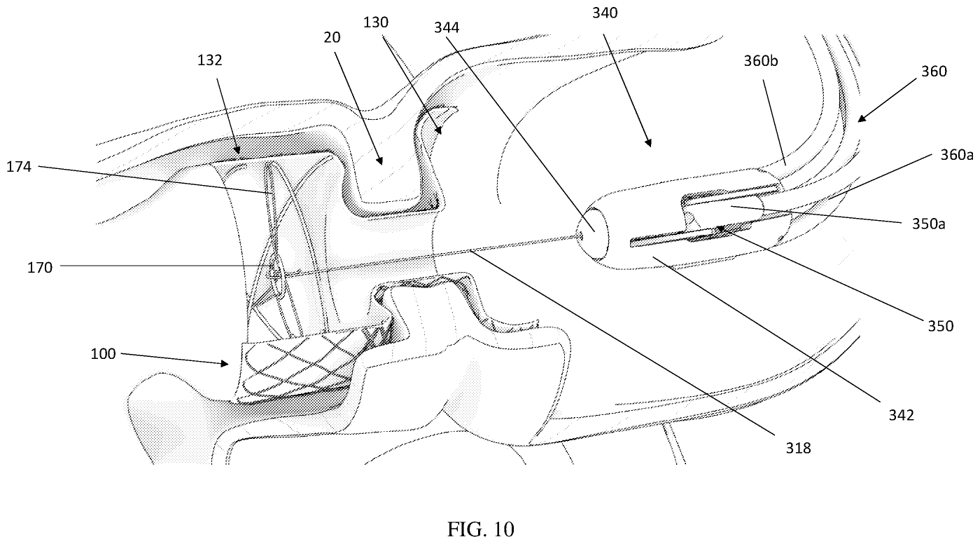

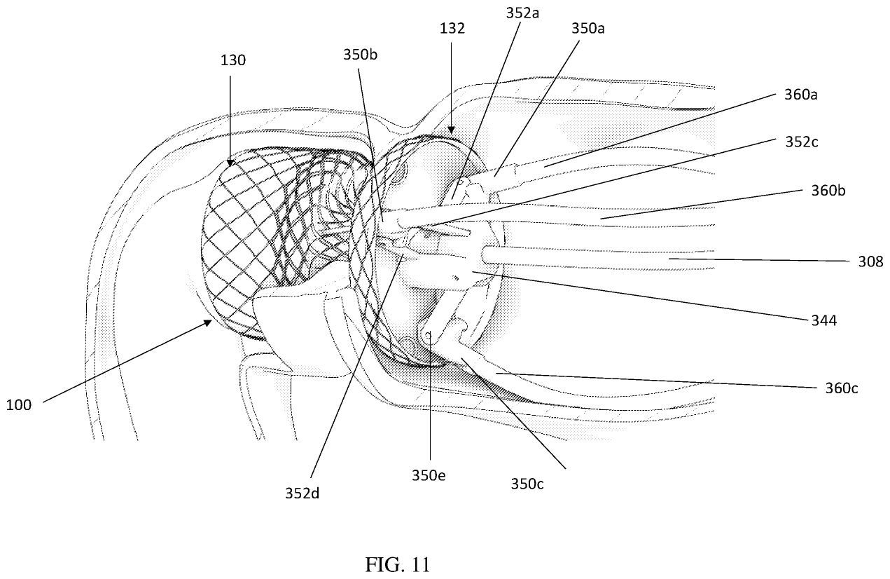

As illustrated in FIG. 10 and FIG. 11 of the present disclosure, the locator system 300 further includes a plurality of extendable arms 350, including a first extendable arm 350a, a second extendable arm 350b, and a third extendable arm 350c, wherein each of which can be selectively deployed to help facilitate a delivery of the suture tethers 200. In various examples, the first, second, and third extendable arms 350a, 350b, 350c are longitudinally spaced apart from one another such that a gap is defined between the first, second, and third extendable arms 350a, 350b, 350c when the first, second, and third extendable arms 350a, 350b, 350c are radially extended from the locator capsule 342. In various examples, the first, second and third extendable arms 350a, 350b, 350c are positioned approximately 120 degrees from one another around a circumference of the head portion 344. Additionally, the locator system 340 is configured such that the first, second and third extendable arms 350a, 350b, 350c are operable to be radially extended from the locator capsule 342, the mechanism of which will be described further herein.

The locator system 340 includes the plurality of linkage arms 352. As illustrated in at least FIG. 11, each the first, second and extendable arms 350a, 350b, 350c includes at least two linkage arms coupled to the extendable arm. For example, the first extendable arm 350a is coupled to a first linkage arm 352a and a second linkage arm 352b, the second extendable arm 350b is coupled to a third linkage arm 352c and a fourth linkage arm 352d, and the third extendable arm 350c is coupled to a fifth linkage arm 352e and a sixth linkage arm 352f. Each of the plurality of linkage arms 352 are rotatably coupled to a respective one of the plurality of extendable arms 350 at one end, and rotatably coupled to the locator capsule 342 at the other end. Such a configuration provides that the delivery system 300 is transitionable between a delivery or collapsed configuration (e.g., where the plurality of extendable arms 350 are stowed within the locator capsule 342, as illustrated in FIG. 10) and a deployment or extended configuration (e.g., where the plurality of extendable arms 350 radially project from the locator capsule 342, as illustrated in FIG. 11). While in the embodiments described the plurality of linkage arms 352 include six linkage arms 352a-f, there may be embodiments where fewer or more linkage arms are desired and included. As shown in FIG. 10, the plurality of linkage arms 352 are also stowed within the locator capsule 342 in the delivery configuration. In contrast, as shown in FIG. 11, the plurality of linkage arms 352 radially extend from the locator capsule 342 while in the deployment configuration. The delivery configuration helps provide that the delivery system 300 maintains a minimal delivery profile.

Additionally, each of the plurality of extendable arm members 350 are attached to one of a plurality of elongate elements 360. For example, the first extendable arm 350a is attached to a first elongate element 360a, the second extendable arm 350b is attached to a second elongate element 360b, and the third extendable arm 350c is attached to a third elongate element 360c. In various examples, each of the plurality of elongate elements 360 includes a lumen extending therethrough. In various embodiments, at least a first needle 362a is coaxially received within the lumen of one of the plurality of elongate elements 360. In various embodiments, the locator system 340 includes a second needle and a third needle (not shown), such that each of the plurality of elongate elements 360 receives one of a plurality of needles.

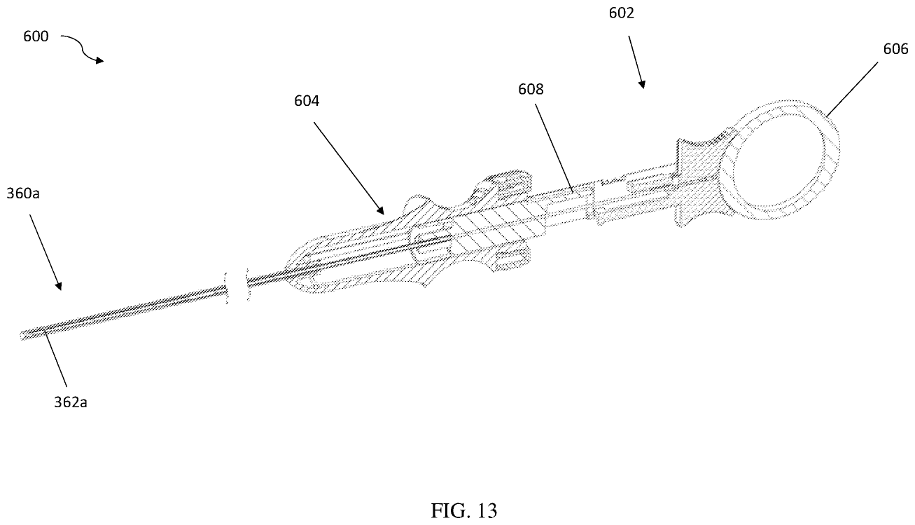

The following description will be disclosed with reference to the first needle 362a but may be applied to any one of the plurality of needles. As discussed in greater detail below, in some examples, the suture tether 200 is situated within the lumen of the needle 362a. In various examples, the first needle 362a is operably coupled an external needle pushing element (not shown), as will be described further with reference to FIG. 13. In some examples, the first needle 362a may be formed from a hollow elongate element having a proximal end and a distal end, wherein the hollow elongate element forming the first needle 362a extends through the lumen of the first elongate element 360a. In some such examples, the proximal end of the elongate element forming the first needle 362a is coupled to the external needle pushing element, and the distal end of the elongate element forming the first needle 362a is configured as a sharp tip suitable for piercing and driving through tissue. In other examples, the first needle 362a may be coupled to a member that extends through the first elongate element 360a. In various embodiments, the distal end of the first needle 362a is rigid and long enough to hold the suture tether 200 in a collapsed configuration during its delivery. The properties of the first needle 362a are such that the proximal section possesses sufficient flexibility to bend during navigation through the anatomy, while the distal portion possesses sufficient stiffness to allow the user to push the distal end of the needle through the target tissue. In some examples, the proximal section of the first needle 362a may be constructed of a high durometer polymer, such as PEEK, Nylon, and polyurethane. The stiffer distal section of the needle may be made from stainless steel, Nitinol, or similar metals which are biocompatible and can be processed to include a sharp tip for piercing tissue. It is to be appreciated, however, that the full length of the first needle 362a may be formed from a single piece of Nitinol, or a length of stainless steel tubing that has been modified in such a way as to make the proximal portion thereof sufficiently flexible to navigate the anatomy. In some such examples, the full length of the needle may be formed from a stainless steel tube that has been laser cut along a portion of its length (e.g., along the proximal portion) in a spiral configuration.

In various examples, an actuation of the external needle pushing element causes a corresponding actuation of the first needle 362a. When actuated, the first needle 362a generally translates (e.g., proximally or distally) relative to the first elongate element 360a, as discussed in greater detail below. In some examples, the external needle pushing element can be actuated to transition the first needle 362a between stowed and deployed states. In the stowed stated, the first needle 362a is stowed within or otherwise concealed within the first elongate element 360a.

In the deployed state, the first needle 362a extends from the first elongate element 360a such that the distal tip of the needle is positioned distal of the first elongate element 360a. As the external needle pushing element is transitioned from the delivery position to the deployed position (e.g., as the external needle pushing element is distally advanced), the first needle 362a translates distally relative to the first elongate element 360a.

Referring again to FIG. 10, the interaction between the gastrointestinal device 100 and the delivery system 300 are detailed further herein. The proximal portion 130 of the device is generally located on the side of the pylorus 20 adjacent the stomach (FIG. 3), with the distal portion 132 generally located on the side of the pylorus 20 generally adjacent the duodenum (FIG. 3). In certain embodiments, the gastrointestinal device 100 additionally includes a suture ring 174 that includes a hoop 170 positioned within the center of the suture ring 174. The suture ring 174 may be connected to the gastrointestinal device 100 in at least three locations such that the hoop 170 is positioned in the center of the through-lumen of the gastrointestinal device 100. As further illustrated in FIG. 10, the guidewire 318 may comprise a looped portion, or coupling element, that is capable of attaching to, or latching onto, the hoop 170 of the gastrointestinal device 100. The connection between the guidewire 318 and the hoop 170 provides for additional stabilization and tracking of the locator system 340 as it travels within the patient's anatomy to be positioned within the gastrointestinal device 100.

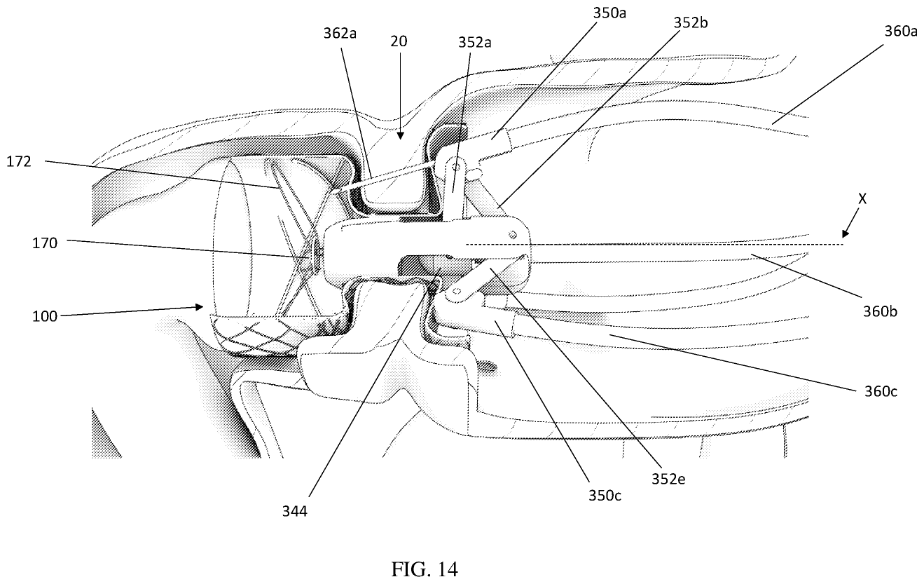

Referring again to the deployed state of FIG. 11, the plurality of extendable arms 350 are relatively extended and positioned against the gastrointestinal device 100. In this configuration, the first, second and third extendable arms 350a, 350b, 350c are stabilized against the gastrointestinal device 100. The first, second and third extendable arms 350a. 350b, 350c are capable of rotation from the retained position illustrated in FIG. 10 to the deployed position in illustrated in FIG. 11. The actuation of the plurality of extendable arms 350 in the stowed position illustrated in FIG. 10, to the deployed position of the extendable arms 350 as shown in FIG. 11, is through the rotation of the second knob 324 of the handle 302, which causes translation of the carriage 332 along the longitudinal axis X. The carriage 332 is operatively coupled to the head portion 344 through the outer catheter 311 of catheter 308 which causes a translation of the head portion 344, as will be described further with reference to FIGS. 12 and 14.

FIG. 12 is a cross-sectional view of the locator system 340 in the deployed state as illustrated in FIG. 11. As illustrated, the plurality of extendable arms 350 are relatively extended such that they could be positioned against the gastrointestinal device (not shown). In this configuration, the head portion 344 has been translated along the longitudinal axis X (FIG. 9) such that the plurality of linkage arms 352 are rotated outward. The rotation and extension of the plurality of linkage arms 352 cause the deployed positioning of the plurality of extendable arms 350. Though the plurality of the extendable arms 350 are in the deployed configuration, the first needle 362a of the first extendable arm 350a has not been deployed and is illustrated retained within the first elongate element 360a of the first extendable arm 350a. The actuation of the external needle pushing element (not shown) may be used to deploy the first needle 362a from this configuration to an advanced configuration, as will be described further with reference to FIG. 13.

FIG. 13 is a cross-sectional view of the external needle pushing element 600. As previously described, the external needle pushing element 600 is used in combination with the plurality of elongate elements 360 to actuate each of the plurality of needles 362 such that the suture tether 200 (FIG. 4A) can be deployed into the tissue of the pylorus 20. As illustrated, the external needle pushing element 600 includes an opening for receiving the distal end of the first needle 362a. The external needle pushing element 600 comprises a handle 602 having an actuator 606 and a body portion 608. The handle 602 is coupled to and positioned within a housing 604. The handle 602 is actuated by the operator through translating the actuator 606 such that it extends distally. As the actuator 606 is translated, the body portion 608 advances distally with reference to the housing 604. This translation advances the first needle 362a through the first elongate element 360a and out of the first extendable arm 350a (FIG. 12) such that the first needle 362a advances into the pylorus 20 (FIG. 12) and through the tissue. The suture tether 200 (FIG. 4A) can then be deployed and secured within the tissue. The operator may then use the external needle pushing element 600 to retract the first needle 362a back into the first elongate element 360a. The deployment process using the external needle pushing element 600 can be repeated with the second and third elongate elements 360b, 360c and the second and third needles 362b, 362c for deploying a plurality of the suture tethers 200.

FIG. 14 is an additional perspective view of the delivery system 300 with the locator system 340 in a deployed configuration, as illustrated in FIGS. 11 and 12. The head portion 344 translates along the longitudinal axis X of the locator system 340 as a result of the translation of the carriage 332. As the head portion 344 translates along the longitudinal axis X, the longitudinal X axis being parallel to a central axis through the lumen of the gastrointestinal device, tension is applied through the connection of the hoop 170 of the suture ring 174 to the guidewire 318. The tension through the guidewire 318 resulting from the connection to the hoop 170 of the suture ring 174 provides additional stabilization of the delivery system 300 while in the deployed position. In embodiments, there is a maximum tension that can be applied through the guidewire 318 such that the operator is prevented from dislodging the gastrointestinal device 100 during the translation of the head portion 344 along the longitudinal axis X. In some examples, this maximum tension value ranges between 15 and 25 N. In one embodiment, the maximum tension value is 22.24 N.

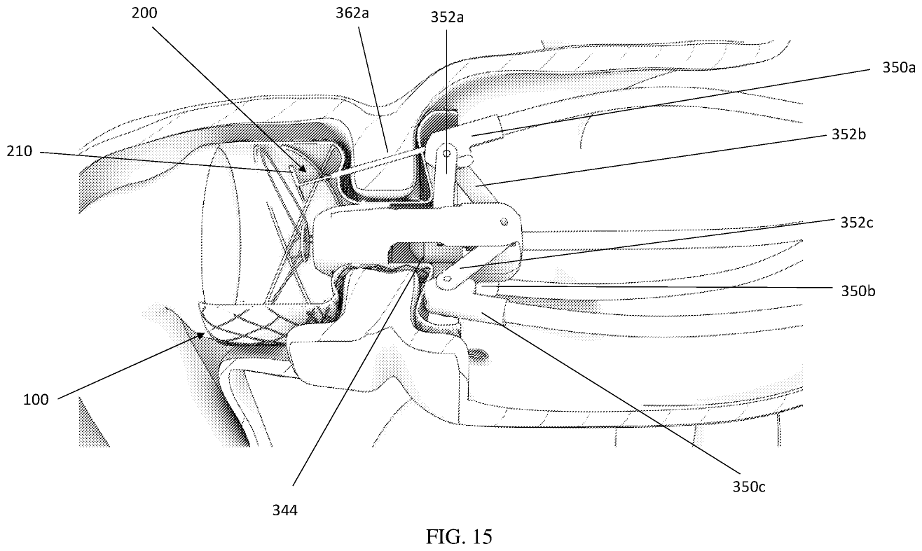

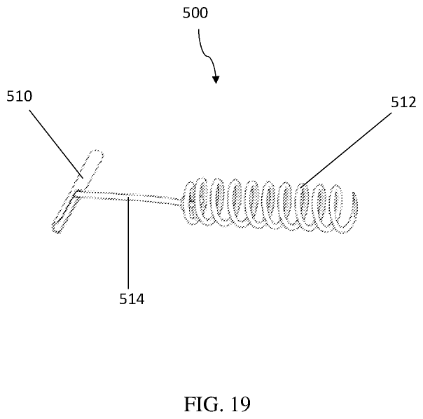

FIGS. 14 and 15 illustrate an advancement of the needle 362a and a deployment of the suture tether 200 from the first elongate element 360a of the first extendable arm 350a. It should be noted that while the following disclosures are described with reference to the suture tether 200, the delivery system 300 may be used with an alternate embodiment of an anti-migration device, an additional example of which is described with reference to FIG. 19. FIG. 14 illustrates the needle 362a advanced from the elongate element 360a such that the needle 362a pierces through the tissue of the pylorus 20. FIG. 15 illustrates the first needle 362a partially retracted to reveal the suture tether 200 with the first retaining tab 210 of the suture tether 200 deployed. Thus, as shown in FIGS. 14 and 15, the first needle 362a (and thus the suture tether 200) can be advanced from a position proximal to the proximal wall flange 148 of a deployed gastrointestinal device 100 to a position distal to the distal wall flange 150 of the deployed gastrointestinal device (FIG. 3). The advancement of the first needle 362a and deployment of the suture tether 200 may be repeated with the second and third elongate elements 360b, 360c of the second and third extendable arms 360a, 360b, respectively. In this way, the operator may advance the first needle 362a, and a second and third needle (not shown), simultaneously or sequentially. If deployed sequentially, the spacing between each extendable arm and their positioning surrounding the gastrointestinal device 100 allows for the deployment of the second and third needles without requiring any repositioning of the gastrointestinal device 100 between the deployment of each needle.

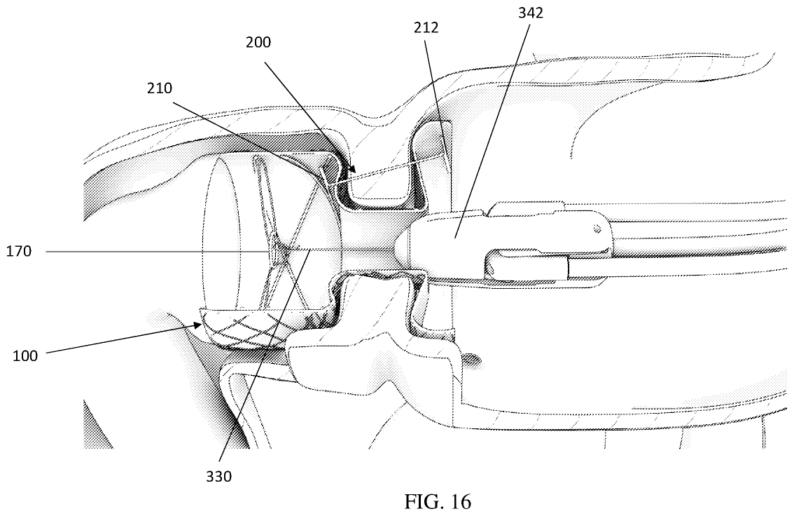

FIG. 16 illustrates the gastrointestinal device 100 delivered within a portion of a gastrointestinal anatomy of a patient with a suture tether 200 deployed. Specifically, as shown in FIG. 16, the suture tether 200 is situated in generally the twelve o'clock position. If the additional needles of the second and third extendable arms 350b, 350c are deployed, the additional suture tethers may be positioned in various other positions around the circumference of the pylorus 20. For example, a suture tether 200 may be situated generally in the three o'clock position, five o'clock position or ten o'clock position. The at least one suture tether 200 extends through the proximal wall flange 148, through the pyloric sphincter 26, and through the distal wall flange 150 at the twelve o'clock position, such that associated first retaining tabs 210 are situated proximal to the proximal wall flange 148, and such that associated second retaining tabs 212 is situated distal to the distal wall flange 150 (FIG. 3).

Thus, it will be appreciated that using the methods described herein, a device, such as the gastrointestinal device 100 shown in FIG. 1, can be secured to the surrounding anatomy via a secondary anchoring mechanism, such as the anti-migration device, so as to minimize a potential for dislodgment and migration. In various embodiments, using the methods described herein, the suture tethers 200 and the gastrointestinal device 100 can be removed from within the patient without using surgery, for example, without forming an incision into the body of the patient.

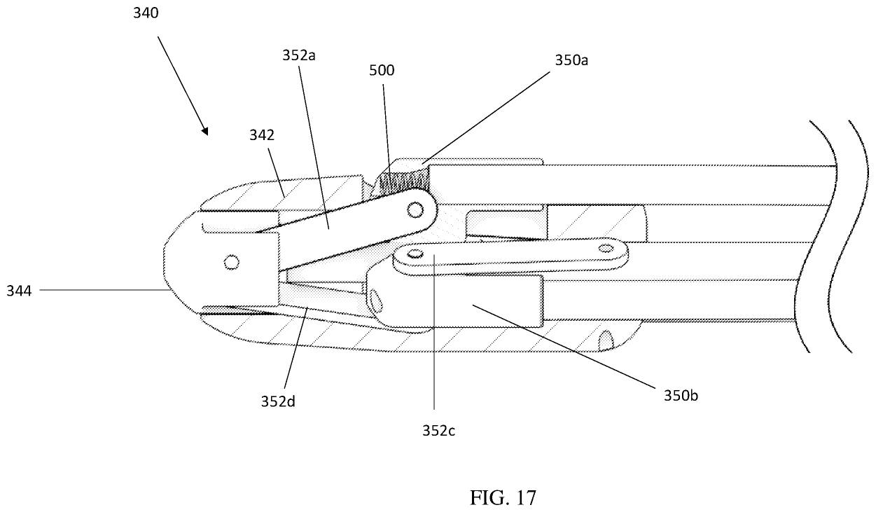

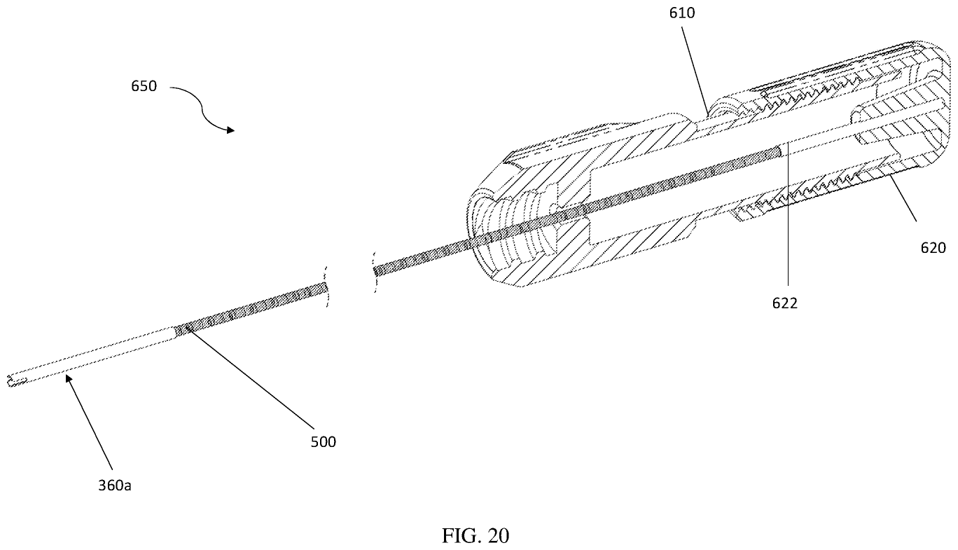

FIG. 17 is a perspective cut away view of an additional embodiment of the locator system 340. The locator system 340 includes the locator capsule 342 and the head portion 344. The locator capsule 342 supports the first extendable arm 350a, the second extendable arm 350b, and the third extendable arm (not shown). The first extendable arm 350a, second extendable arm 350b, and third extendable arm each comprise the first elongate element 360a, second elongate element 360b and third elongate element (not shown), respectively. As described previously, each elongate element 360a. 360b, 360c may comprise a lumen extending therethrough. Within that lumen a helix element or helix anchor 500 may be housed. The helix element 500 is deployed with the use of an external helix pushing element, as will be described further in reference to FIG. 20.

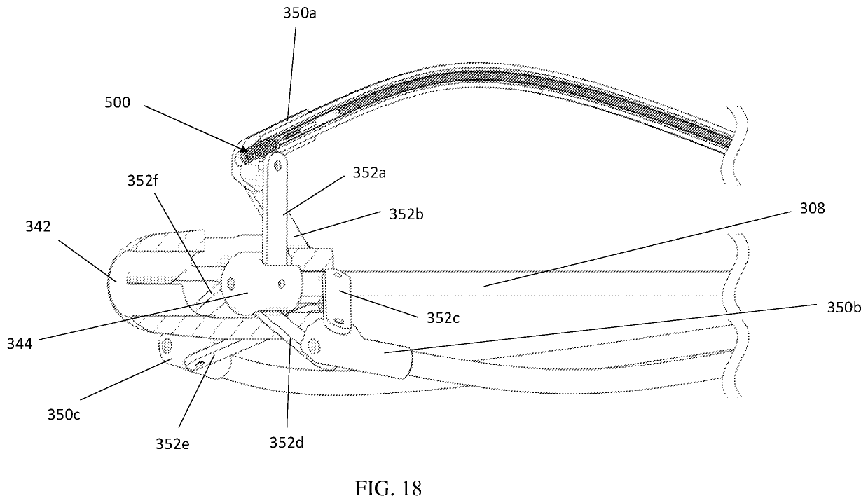

FIG. 18 illustrates the delivery system 300 in a deployed positioned omitting the patient's anatomy. Each the first, second and third extendable arms 350a, 350b, 350c are shown in a radially extended positioned with respect to the locator capsule 342. Similar to the descriptions with reference to FIG. 13, the head portion 344 has translated along the longitudinal axis X to a retracted position in the deployed configuration. In this embodiment, the one or more helix elements 500 can be deployed into the pyloric tissue from the proximal side of the gastrointestinal device 100 (FIG. 5). The helix element 500 provides additional stabilizing through the anchoring of the gastrointestinal device 100 (FIG. 5) to the tissue of the pylorus. The helix element 500 will be described in further detail with reference to FIG. 19.

Turning now to FIG. 19, in some embodiments, the helix element 500 is an anti-migration device that includes a body 512 having a retaining tab 510. The helix element 500 may have a connecting portion 514 positioned between and coupled to the body 512 and the retaining tab 510. Similar to the previously described suture tether 200, the retaining tab 510 may have one or more features to help facilitate a minimal delivery profile. For example, the retaining tab 510 may be accommodated within the body 512 of the helix element 500, such as is illustrated with reference to the suture tether 200 in FIG. 4A to aid in the minimal delivery. In the deployed configuration and once the helix element 500 is to be secured, the retaining tab 510 may transition to the deployed configuration of the retaining tab 210 as illustrated with the suture tether 200 in FIG. 4B. The retaining tab 510 provides an anchoring of the helix element 500. In various embodiments, the helix element 500 does not comprise the connecting portion 514 or the retaining tab 510 and only includes the body 512. In these embodiments, the body 512 is deployed into the pyloric tissue from the proximal side 130 and punctures the pyloric tissue. As such, the anchoring of the gastrointestinal device 100 is only completed on the proximal side 130 of the gastrointestinal device 100 (FIG. 3). In the instances wherein the helix element 500 does not comprise the connecting portion 514 nor the retaining tab 510, the helix element 500 may be permanent and remain in the patient's tissue.

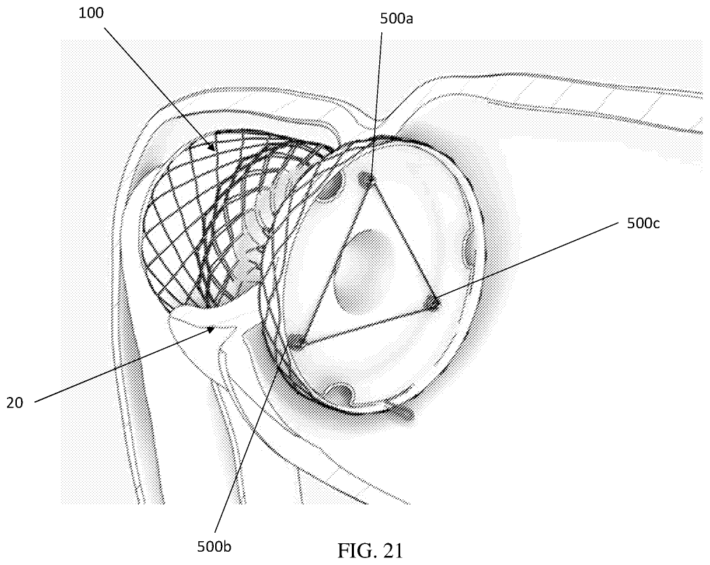

FIG. 20 is a cross-sectional side view of an external helix pushing element 650 for deploying the helix element 500. In this embodiment, the external helix pushing element 650 comprises a housing 610 operatively coupled to a knob 620. Extending through and within the housing 610 is a lumen 622 for receiving the helix element 500. The lumen 622 is configured for attachment with one of the plurality of elongate elements 360. For example, the lumen 622 is receives the first elongate element 360a. The knob 620 comprises an internal component 624 wherein the lumen 622 is received and fixed to the knob 620. The operator can actuate the knob 620 through rotation of the knob 620. Rotation of the knob 620 causes rotation of the lumen 622 and couples to the helix element 500 such that the helix element 500 rotates as well. This advances the helix element 500 distally through the lumen 622 and the first elongate element 360a. The knob may be rotated until the helix element 500 advances out of the first extendable arm 350a (FIG. 18) and into the pyloric tissue of the patient. As the helix element 500 is rotated through the operation of the external helix pushing element 650, the helix element 500 rotates to pierce the pyloric tissue and advance into the tissue and thus secures itself within the tissue. This deployed configuration of the helix element 500 will be described further herein with reference to FIG. 21.

FIG. 21 illustrates the gastrointestinal device 100 delivered within a portion of a gastrointestinal anatomy of a patient with a plurality of the helix element 500 deployed. In this embodiment, a first helix element 500a, a second helix element 500b, and a third helix element 500c has been deployed. In various embodiments, a larger or smaller number of helix elements 500 may be used to secure the gastrointestinal device 100. The configuration of the helix elements 500a-c is such that the proximal portion 130 (FIG. 3) of the gastrointestinal device 100 is secured to the pylorus 20 since the helix elements 500a-c do not extend all the way through the tissue of the pylorus 20. Although, in various other embodiments, each helix element 500a-c may extend through the tissue of the pylorus 20 completely such that it comprises a portion in the distal portion 132 (FIG. 3) of the gastrointestinal device 100.

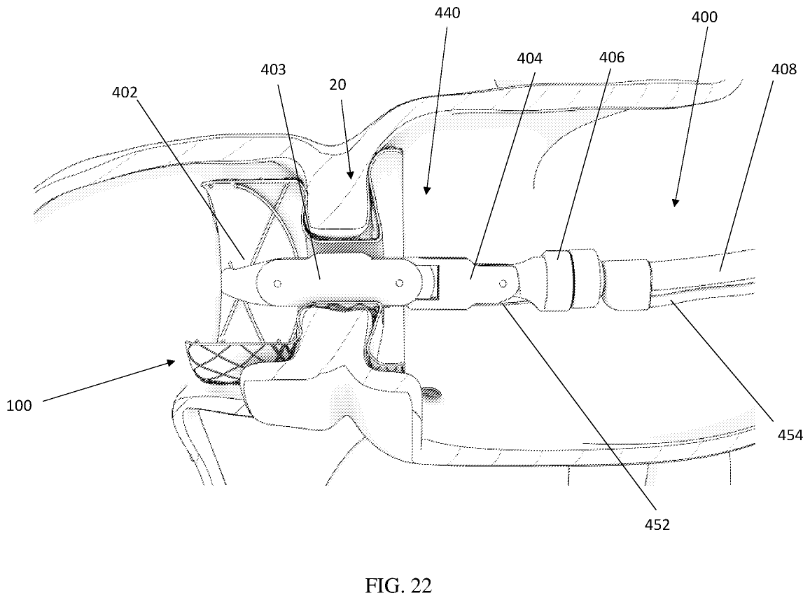

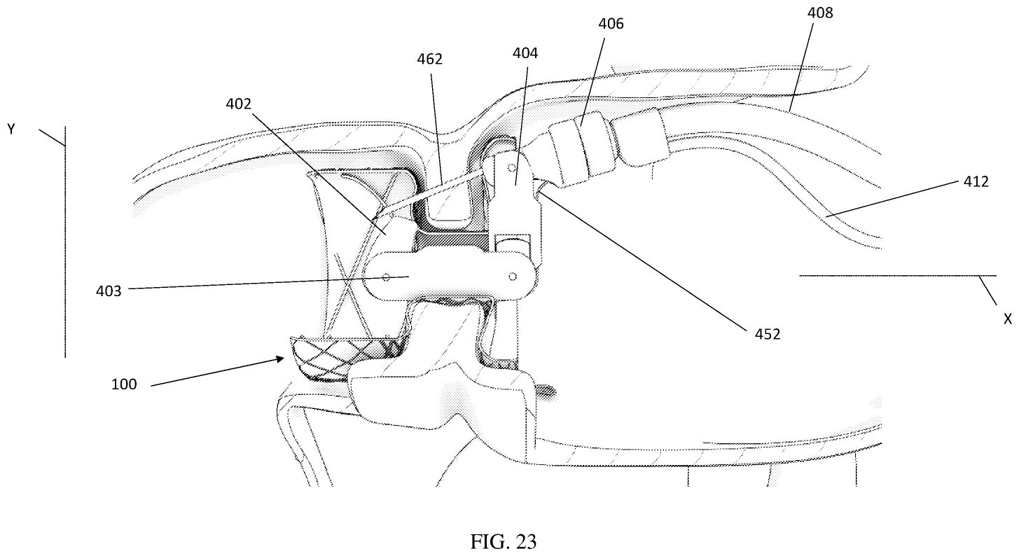

FIG. 22 is a cross-sectional side view of a portion of an additional embodiment of a delivery system 400 comprising a locator system 440. In some embodiments, the delivery system 400 may comprise the handle 302 as illustrated and disclosed with reference to FIGS. 6-10. The handle 302 may be used in conjunction with the locator system 440 in a similar manner as described with reference to locator system 340. The delivery system 400 includes an elongate element 408 having a distal portion 410, a connecting or linking element 406 adjacent the elongate element 408, a first proximal chain element 404 rotatably coupled to the linking element 406, a second intermediate chain element 403 rotatably coupled to the first chain element, and a third distal chain element 402 rotatably coupled to the second chain element 403. While described as the first, second and third chain elements 404, 403, and 402, they may also be referred to as the first, second and third linkages 404, 403, and 402, respectively. In some embodiments, a pin is used to rotatably couple the first and second chain elements 404, 403 and the second and third chain elements 403, 402. In other embodiments, the rotatable coupling may be accomplished using a hinge assembly. The delivery system 400 further includes a catheter 454 including a tensioning element 452 which may extend through the catheter 454. In other embodiments, the tensioning element 452 extends through the elongate element 408. In some embodiments, the tensioning element 452 is a suture. In other embodiments, the tensioning element 452 is a wire. The tensioning element 452 may extend through the linking element 406, the first chain element 404, the second chain element 403, and the third chain element 402. In other instances, the tensioning element 452 just extends through the linking element 406, the first chain element 404, and the second chain element 403. The configuration of the delivery system 400 in FIG. 20 is shown in a delivery configuration. The operator may actuate the delivery system 400 in order to transition the delivery system 400 into a deployed configuration, which is illustrated and described further herein with reference to FIG. 23.

FIG. 23 is a cross-sectional side view of a portion of the embodiment of the delivery system 400 illustrated in FIG. 22. The delivery system 400 is positioned within a portion of the gastrointestinal anatomy of a patient such that the device extends through the lumen of the gastrointestinal device 100. FIG. 23 illustrates the gastrointestinal device 100 during the deployment of an anti-migration device, such as the suture tether 200 described previously. The operator is able to actuate the device to apply tension onto the tensioning element 452 and cause rotation of the first, second and third chain elements 404, 403, 402. This applied tension onto the tensioning element 452 may be applied through rotation of a knob of the handle 302 (FIG. 7). When this rotation and applied tension occurs, the first, second, and third chain elements 404, 403, 402 move from their relatively longitudinal positioning to a generally C-shaped positioning as illustrated in FIG. 23. This is defined by the first chain element 404 being positioned relatively parallel with an axis Y, the second chain element 403 retained in a position relatively parallel to the longitudinal axis X, and the third chain element 402 positioned relatively parallel with axis Y. The first and third chain elements 404, 402, thus cooperatively compress the tissue of the pylorus 20 which may increase stabilization of the device before deployment of the anti-migration device. This also ensures the correct positioning of the anti-migration device when deployed as the delivery system 400 is stabilized against the gastrointestinal device 100. The compression of the pylorus 20 may be limited by a couple of factors. Firstly, the tensioning element 452 has a maximum tension that may be applied and once the maximum tension value has been reached, the rotation of the first knob 320 is stopped and the first and third chain elements 404, 402 will not compress the pylorus 20 any further. In various embodiments, the maximum tension value may range from 15 to 25 N. In various embodiments, the maximum tension value is 22.24 N. Additionally, as illustrated in FIG. 23, the pivoting motion of the first chain element portion 404 while compressing the pylorus 20 is limited through the contact with a portion of the linking element 406. Thus, the first chain element 404 is only able to rotate a certain angle before any further motion is blocked through the linking element 406 and the first chain element 404 contact.

The locator system 440 additionally includes a needle 462 that includes a lumen extending through it wherein through which extends the anti-migration device, such as the suture tether 200 (FIG. 15). In various embodiments, the needle 462 is integrated within the locator system 440 and is received within the elongate element 408. In other embodiments, the needle 462 may be actuated through the use of an external needle pushing element, such as the external needle pushing element 600 (FIG. 13), wherein the external needle pushing element 600 is used in conjunction with catheter 412, or the elongate element 408, of the delivery system 400 to advance the needle 462. In these various embodiments, the operator can actuate the deployment of the suture tether 200 into the pylorus 20 and secure the gastrointestinal device 100. In these embodiments, more than one suture tether 200 may be deployed, but in these embodiments, it would require deployment of the suture tethers 200 sequentially, rather than simultaneously. After deployment of one suture tether 200, the first, second, and third chain elements 404, 403, 402 are returned to the positioning as illustrated in FIG. 22. In various instances, the elongate element 408 includes a guidewire (not shown) that may allow for the return of the chain elements 404, 403, 402 to the extended position in FIG. 22, once the operator applied tension on the tensioning element 452 is released. In embodiments, this is due to the guidewire having a predetermined tension that causes a passive return of the chain elements 404, 403, 402 to the extended position when the tension on the tensioning element 452 is released.

Following the repositioning of the first, second, and third chain elements 404, 402, 402, the locator system 440 may be rotated within the lumen of the gastrointestinal device 100 at an angle. The angle may range from 10 degrees to 350 degrees. In various embodiments, the angle may range from 100 to 200 degrees. In preferred embodiments, the angle may be 180 degrees. After the rotation of the locator system 440, the operator may actuate the locator system 440 again such that the first, second, and third chain elements 404, 403, 402 are rotated back into the positioning illustrated in FIG. 22. The first, second and third chain elements 404, 403, 402 can again compress the together the tissue of the pylorus 20 and stabilize the locator capsule 442. In embodiments, the operator may then deploy and additional anti-migration device, such as the suture tether 200. In various embodiments, this is deployment of an additional anti-migration device may be completed several times.

While the above discussed examples are illustrated and described with regard to a gastrointestinal device used in association with the pylorus, the devices, systems, and methods discussed herein may be utilized in other anatomical areas without departing from the spirit or scope of the present disclosure. Thus, the examples illustrated and described above should not be interpreted as limiting.

Numerous characteristics and advantages have been set forth in the preceding description, including various alternatives together with details of the structure and function of the devices and/or methods. Moreover, the inventive scope of the various concepts addressed in this disclosure has been described both generically and with regard to specific examples. The disclosure is intended as illustrative only and as such is not intended to be exhaustive. For example, the various embodiments of the present disclosure are described in the context of medical applications but can also be useful in non-medical applications. It will be evident to those skilled in the art that various modifications may be made, especially in matters of structure, materials, elements, components, shape, size, and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

1. A system for delivering an anti-migration anchor to a gastrointestinal device including a proximal portion, a distal portion and a neck portion, the anti-migration anchor including a first end for contacting the proximal portion and a tether portion for extending through a pylorus, the delivery system comprising:
a locator system having a head portion adapted to mate with the neck portion of the gastrointestinal device, a locator capsule coupled to the head portion, and a plurality of arms coupled to the head portion and the locator capsule, the arms having a collapsed configuration for delivery to the pylorus and an extended configuration for deployment of the anti-migration anchor,
a catheter having a distal portion coupled to and extending from the head portion, the catheter including an outer member coupled to the head portion and an inner member coupled to the capsule;
a handle coupled to a proximal portion of the catheter, the handle having a first knob configured to engage a guidewire and a second knob configured to effect relative motion between the inner member and the outer member of the catheter, so as to cause the arms to transition from the collapsed configuration to the extended configuration; and
a plurality of elongate elements having a proximal end and a distal end coupled the locator system arm, the elongate elements configured to facilitate the delivery of an anti-migration anchor to the locator system arm;
wherein each of the locator system, the catheter, and the handle are configured to be advanced over a guidewire.

2. The system of claim 1 further comprising a guidewire extending through each of the locator system, the catheter, and the handle, the guidewire including a distal coupling element for coupling to the gastrointestinal device.

3. The system of claim 2 wherein rotation of the first knob causing translation of the delivery system with respect to the guidewire.

4. The system of claim 3 wherein the handle further comprising a locking mechanism to lock the position of the handle with respect to the guidewire.

5. The system of claim 1 wherein the locator system includes three arms, each angularly spaced at an angle of about 120 degrees about a circumference of the head portion.

6. The system of claim 1 further comprising a deployment element configured to be delivered through one of the plurality of elongate elements to the locator system arm.

7. The system of claim 6 wherein the deployment element is a needle pushing mechanism adapted to advance a needle element forward through the pylorus and through the distal portion of the gastrointestinal device.

8. The system of claim 7 further comprising a suture tether coupled inside the needle element and configured to deploy after the needle element is advanced through the pylorus.

9. The system of claim 8 wherein the suture tether includes a first retaining tab for coupling with the proximal portion of the gastrointestinal device and a second retaining tab for coupling with the distal portion of the gastrointestinal device, the first and second tabs connected by a flexible tether.

10. The system of claim 6 wherein the deployment element is a helix delivery element adapted to advance a helix anchor into the pylorus.

11. The system of claim 10 wherein the helix anchor includes a helical portion configured to embed within the pylorus and a retaining tab for coupling with the proximal portion of the gastrointestinal device

12. A system for delivering an anti-migration anchor to an implantable device including a proximal portion and a distal portion, the anti-migration anchor including a first end for contacting the proximal portion and a tether portion for extending through a tissue, the delivery system comprising:
a locator system having a head portion adapted to mate with the neck portion of the gastrointestinal device, a locator capsule coupled to the head portion, and a plurality of arms coupled to the head portion and the locator capsule, the arms having a collapsed configuration for delivery and an extended configuration for deployment of the anti-migration anchor;
a catheter having a distal portion coupled to and extending from the head portion, the catheter including an outer member coupled to the head portion and an inner member coupled to the capsule;
a handle coupled to a proximal portion of the catheter, the handle having a mechanism to effect relative motion between the inner member and the outer member of the catheter, so as to cause the arms to transition from the collapsed configuration to the extended configuration; and
a plurality of elongate elements having a proximal end and a distal end coupled the locator system arm, the elongate elements configured to facilitate the delivery of an anti-migration anchor to the locator system arm.

13. The system of claim 12 wherein the implantable device is a gastrointestinal implant and the issue is the pylorus, the implant further including a neck portion adapted to extend through the internal circumference of the pylorus.

14. A system for delivering an anti-migration anchor to a gastrointestinal device including a proximal portion, a distal portion and a neck portion, the anti-migration anchor including a first end for contacting the proximal portion and a tether portion for extending through a pylorus, the delivery system comprising:
a locator system having a proximal linkage and a distal linkage each pivotably coupled to opposite sides of an intermediate linkage, the linkages having a first configuration for delivery to the pylorus and a second configuration for deployment of the anti-migration anchor;
a catheter having a distal portion coupled to and extending from locator system,
a tension element extending through the catheter element and coupled to the distal element of the locator system;
a handle coupled to a proximal portion of the catheter, the handle having a mechanism to apply a tension to the tension element, so as to cause the locator system to transition from the first configuration to second configuration; and
an elongate element having a proximal end and a distal end coupled the proximal linkage of the locator system, the elongate element configured to facilitate the delivery of an anti-migration anchor to the locator system.

15. The system of claim 14 further comprising a deployment element configured to be delivered through the elongate element to the proximal linkage.

16. The system of claim 15 wherein the deployment element is a needle pushing mechanism adapted to advance a needle element forward through the pylorus and through the distal portion of the gastrointestinal device.

17. A method for delivering an anti-migration anchor to an implantable device including a proximal portion and a distal portion, the anti-migration anchor including a first end for contacting the proximal portion and a tether portion for extending through a pylorus, the method comprising:
advancing a delivery system over a guidewire to the pylorus, the delivery system including a locator having a head portion adapted to mate with the neck portion of the gastrointestinal device, a locator capsule coupled to the head portion, and a plurality of arms coupled to the head portion and the locator capsule, the arms having a collapsed configuration for delivery and an extended configuration for deployment of the anti-migration anchor, a catheter having a distal portion coupled to and extending from the head portion, the catheter including an outer member coupled to the head portion and an inner member coupled to the capsule, a handle coupled to a proximal portion of the catheter, the handle having a mechanism to effect relative motion between the inner member and the outer member of the catheter, so as to cause the arms to transition from the collapsed configuration to the extended configuration, and a plurality of elongate elements having a proximal end and a distal end coupled the locator system arm, the elongate elements configured to facilitate the delivery of an anti-migration anchor to the locator system arm;
manipulating the mechanism on the handle to cause the arms to deploy from the collapsed configuration to the extended configuration;
advancing the anti-migration anchor through the elongate element to the locator adjacent the pylorus; and
advancing the anti-migration anchor out of the delivery system and through the pylorus, such that the anti-migration system deploys;
wherein the anti-migration system includes a includes a first retaining tab for coupling with the proximal portion of the gastrointestinal device and a second retaining tab for coupling with the distal portion of the gastrointestinal device, the first and second retaining tabs connected by a flexible tether.
